SEQUENCE LISTING (48 sequences)

SEQ ID NO: 1 - RNA, 21 nt, synthetic construct
  source 1..21: mol_type = other RNA
caggccttcagtcagtccttt

SEQ ID NO: 2 - RNA, 21 nt, synthetic construct
  source 1..21: mol_type = other RNA
atccatgcaggccttcagtca

SEQ ID NO: 3 - RNA, 21 nt, synthetic construct
  source 1..21: mol_type = other RNA
aatccatgcaggccttcagtc

SEQ ID NO: 4 - RNA, 21 nt, synthetic construct
  source 1..21: mol_type = other RNA
atggaatccatgcaggccttc

SEQ ID NO: 5 - RNA, 21 nt, synthetic construct
  source 1..21: mol_type = other RNA
aacatggaatccatgcaggcc

SEQ ID NO: 6 - RNA, 21 nt, synthetic construct
  source 1..21: mol_type = other RNA
gaacatggaatccatgcaggc

SEQ ID NO: 7 - RNA, 21 nt, synthetic construct
  source 1..21: mol_type = other RNA
atgaacatggaatccatgcag

SEQ ID NO: 8 - RNA, 21 nt, synthetic construct
  source 1..21: mol_type = other RNA
tcatgaacatggaatccatgc

SEQ ID NO: 9 - RNA, 21 nt, synthetic construct
  source 1..21: mol_type = other RNA
aactcatgaacatggaatcca

SEQ ID NO: 10 - RNA, 21 nt, synthetic construct
  source 1..21: mol_type = other RNA
tctttgtcagcagtcacattg

SEQ ID NO: 11 - RNA, 21 nt, synthetic construct
  source 1..21: mol_type = other RNA
tcggccacaccatctttgtca

SEQ ID NO: 12 - RNA, 21 nt, synthetic construct
  source 1..21: mol_type = other RNA
tcttcaatagacacatcggcc

SEQ ID NO: 13 - RNA, 21 nt, synthetic construct
  source 1..21: mol_type = other RNA
tggaccaccagtgtgcggcca

SEQ ID NO: 14 - RNA, 21 nt, synthetic construct
  source 1..21: mol_type = other RNA
aattacaccacaagccaaacg

SEQ ID NO: 15 - RNA, 21 nt, synthetic construct
  source 1..21: mol_type = other RNA
cagtttctcactacaggtact

SEQ ID NO: 16 - RNA, 21 nt, synthetic construct
  source 1..21: mol_type = other RNA
ggcaaaatacaggtcattgaa

SEQ ID NO: 17 - RNA, 60 nt, synthetic construct
  source 1..60: mol_type = other RNA
gaaggactgaataaaggccactggagtgagtagcaggtccaggccttcagtcagtccttt

SEQ ID NO: 18 - RNA, 60 nt, synthetic construct
  source 1..60: mol_type = other RNA
tgattgaaggatagcatggacatgagtgagtagcaggtcatccatgcaggccttcagtca

SEQ ID NO: 19 - RNA, 60 nt, synthetic construct
  source 1..60: mol_type = other RNA
ggttgaaggcatacatggaccttgagtgagtagcaggtcaatccatgcaggccttcagtc

SEQ ID NO: 20 - RNA, 60 nt, synthetic construct
  source 1..60: mol_type = other RNA
ggaggcctgcatagattccacatgagtgagtagcaggtcatggaatccatgcaggccttc

SEQ ID NO: 21 - RNA, 60 nt, synthetic construct
  source 1..60: mol_type = other RNA
ggtttgcatgatatccatgccttgagtgagtagcaggtcaacatggaatccatgcaggcc

SEQ ID NO: 22 - RNA, 60 nt, synthetic construct
  source 1..60: mol_type = other RNA
gtttgcatggataccatgtactcgagtgagtagcaggtcgaacatggaatccatgcaggc

SEQ ID NO: 23 - RNA, 60 nt, synthetic construct
  source 1..60: mol_type = other RNA
ttgtatggatataatgttcacatgagtgagtagcaggtcatgaacatggaatccatgcag

SEQ ID NO: 24 - RNA, 60 nt, synthetic construct
  source 1..60: mol_type = other RNA
gtatggattcatagttcatacgagagtgagtagcaggtctcatgaacatggaatccatgc

SEQ ID NO: 25 - RNA, 60 nt, synthetic construct
  source 1..60: mol_type = other RNA
tgggttccatatacatgagacttgagtgagtagcaggtcaactcatgaacatggaatcca

SEQ ID NO: 26 - RNA, 60 nt, synthetic construct
  source 1..60: mol_type = other RNA
cgatgtgactatagacaaaacgagagtgagtagcaggtctctttgtcagcagtcacattg

SEQ ID NO: 27 - RNA, 60 nt, synthetic construct
  source 1..60: mol_type = other RNA
tgataaagatatagtggccacgagagtgagtagcaggtctcggccacaccatctttgtca

SEQ ID NO: 28 - RNA, 60 nt, synthetic construct
  source 1..60: mol_type = other RNA
ggtcggtgtgataattgaaacgagagtgagtagcaggtctcttcaatagacacatcggcc

SEQ ID NO: 29 - RNA, 60 nt, synthetic construct
  source 1..60: mol_type = other RNA
tggtcgtacaatagtggtcaccagagtgagtagcaggtctggaccaccagtgtgcggcca

SEQ ID NO: 30 - RNA, 60 nt, synthetic construct
  source 1..60: mol_type = other RNA
cgtttggtttatagtgtaaacttgagtgagtagcaggtcaattacaccacaagccaaacg

SEQ ID NO: 31 - RNA, 60 nt, synthetic construct
  source 1..60: mol_type = other RNA
agtatctgtaataagaaacactggagtgagtagcaggtccagtttctcactacaggtact

SEQ ID NO: 32 - RNA, 60 nt, synthetic construct
  source 1..60: mol_type = other RNA
ttcgatgaccataattttgacccgagtgagtagcaggtcggcaaaatacaggtcattgaa

SEQ ID NO: 33 - RNA, 21 nt, Homo sapiens
  source 1..21: mol_type = mRNA
aaaggactgactgaaggcctg

SEQ ID NO: 34 - RNA, 21 nt, Homo sapiens
  source 1..21: mol_type = mRNA
tgactgaaggcctgcatggat

SEQ ID NO: 35 - RNA, 21 nt, Homo sapiens
  source 1..21: mol_type = mRNA
gactgaaggcctgcatggatt

SEQ ID NO: 36 - RNA, 21 nt, Homo sapiens
  source 1..21: mol_type = mRNA
gaaggcctgcatggattccat

SEQ ID NO: 37 - RNA, 21 nt, Homo sapiens
  source 1..21: mol_type = mRNA
ggcctgcatggattccatgtt

SEQ ID NO: 38 - RNA, 22 nt, Homo sapiens
  source 1..22: mol_type = mRNA
gcctgcatggattccatgttca

SEQ ID NO: 39 - RNA, 21 nt, Homo sapiens
  source 1..21: mol_type = mRNA
ctgcatggattccatgttcat

SEQ ID NO: 40 - RNA, 21 nt, Homo sapiens
  source 1..21: mol_type = mRNA
gcatggattccatgttcatga

SEQ ID NO: 41 - RNA, 21 nt, Homo sapiens
  source 1..21: mol_type = mRNA
tggattccatgttcatgagtt

SEQ ID NO: 42 - RNA, 21 nt, Homo sapiens
  source 1..21: mol_type = mRNA
caatgtgactgctgacaaaga

SEQ ID NO: 43 - RNA, 21 nt, Homo sapiens
  source 1..21: mol_type = mRNA
tgacaaagatggtgtggccga

SEQ ID NO: 44 - RNA, 21 nt, Homo sapiens
  source 1..21: mol_type = mRNA
ggccgatgtgtctattgaaga

SEQ ID NO: 45 - RNA, 21 nt, Homo sapiens
  source 1..21: mol_type = mRNA
tggccgcacactggtggtcca

SEQ ID NO: 46 - RNA, 21 nt, Homo sapiens
  source 1..21: mol_type = mRNA
cgtttggcttgtggtgtaatt

SEQ ID NO: 47 - RNA, 21 nt, Homo sapiens
  source 1..21: mol_type = mRNA
agtacctgtagtgagaaactg

SEQ ID NO: 48 - RNA, 21 nt, Homo sapiens
  source 1..21: mol_type = mRNA
ttcaatgacctgtattttgcc

(19) United States
(12) Patent Application Publication
(10) Pub. No.: US 2026/0218194 A1
(43) Pub. Date: Jul. 30, 2026

(54) COMPOSITIONS AND METHODS FOR TREATING NEUROLOGICAL DISEASES

(71) Applicant: King’s College London, London (GB)

(72) Inventors: Christopher SHAW, Camberwell, London (GB); Youn Bok LEE, Camberwell, London (GB); Romain JOUBERT, London (GB); Do Young LEE, London (GB)

(21) Appl. No.: 19/139,060

(22) PCT Filed: Dec. 14, 2023
(86) PCT No.: PCT/EP2023/085847

§ 371 Date: Jun. 13, 2025

Related U.S. Application Data
(60) Provisional application No. 63/432,587, filed on Dec. 14, 2022.

(51) Int. Cl. C12N 15/113; C12N 15/86

(52) U.S. Cl. CPC C12N 15/1137; C12N 15/86; C12Y 115/01001; C12N 2310/141; C12N 2310/531; C12N 2750/14143

(57) ABSTRACT

The disclosure features compositions and methods for the treatment of disorders associated with expression of wild-type or mutant superoxide dismutase 1 (SOD1) RNA transcripts, that can lead to a pathological phenotype. Disclosed herein are inhibitory RNA constructs that suppress the expression of SOD1, as well as viral vectors, such as adeno-associated viral vectors, encoding such inhibitory RNA molecules.

### SEQUENCE LISTING

The instant application contains a Sequence Listing which has been submitted electronically in XML format and is hereby incorporated by reference in its entirety. Said XML copy, created on Nov. 14, 2023, is named “51585-013WO2_Sequence_Listing_11_14_23” and is 43,593 bytes in size.

### FIELD OF THE INVENTION

The invention relates to the field of nucleic acid biotechnology and provides compositions and methods for treating diseases associated with expression of superoxide dismutase 1 (SOD1).

### BACKGROUND OF THE INVENTION

Superoxide dismutase 1 (SOD1) is an enzyme that destroys free superoxide radicals in the body. It is one of the three superoxide dismutases in the human body and binds to copper and zinc. SOD1 enzyme is encoded by the SOD1 gene and is associated with diseases such as amyotrophic lateral sclerosis (ALS). It is implicated in both familial and sporadic ALS and mutations in this gene are the second most frequent cause of familial ALS. Most common mutations found in the SOD1 gene include D90A, A4V, H46R and G93A. A4V is the most common mutation in patients with ALS in the United States. Mutations in the SOD1 gene can lead to oxidative damage in mitochondrial DNA of motor neurons such as, spinal motor neurons. ALS is progressive, neurodegenerative, can be highly debilitating, and is often fatal in nature. It predominantly affects upper and lower motor neurons. There is currently a paucity of strategies available for successfully treating and ameliorating the symptoms of ALS, Parkinson's disease, Huntington's disease, and other diseases or disorders associated with wild-type or mutant SOD1. Accordingly, there remains a need for effective therapeutics for these pathologies.

### SUMMARY OF THE INVENTION

Described herein are compositions and methods useful for treating diseases associated with expression of wild-type or mutant superoxide dismutase 1 (SOD1). The compositions described herein that may be used to treat such disorders include inhibitory nucleic acid constructs, for example, interfering RNA constructs, that suppress the expression of wild-type or mutant mRNA transcripts. Exemplary inhibitory nucleic acids of the disclosure are, without limitation, microRNA (miRNA), short hairpin RNA (shRNA), and short interfering RNA (siRNA) constructs. Without being limited by mechanism, these inhibitory nucleic acids may anneal to portions of wild-type or mutant SOD1 mRNA and promote the degradation of pathological transcripts by way of various cellular processes. The present disclosure additionally features vectors, such as viral vectors, encoding such inhibitory nucleic acid constructs. Exemplary viral vectors described herein that encode inhibitory nucleic acid constructs (such as interfering RNA constructs (e.g., miRNA)) are adeno-associated viral (AAV) vectors, such as pseudotyped AAV2/8 and AAV2/9 vectors.

Using the compositions and methods described herein, a patient diagnosed as having a disease associated with wild-type or mutant SOD1, such as amyotrophic lateral sclerosis (ALS), Huntington's disease, and Parkinson's disease, among others, can be administered an inhibitory nucleic acid, such as an interfering RNA construct, or a vector encoding the same, so as to reduce the expression of wild-type or mutant mRNA transcripts. For example, the compositions and methods described herein can be used to treat patients having ALS, as such patients may be administered an inhibitory nucleic acid construct or a viral vector, such as an AAV vector, encoding such a construct, thereby reducing the expression of mRNA transcripts encoding wild-type or mutated SOD1 protein. Most common mutations found in the SOD1 gene include D90A, A4V, H46R and G93A. A4V is among the most common mutations in patients with amyotrophic lateral sclerosis (ALS) in the United States. The compositions and methods described herein can be used to treat patients expressing wild-type or mutant SOD1 mRNA (for e.g., wild-type or mutant human SOD1 mRNA), e.g., by using inhibitory nucleic acid constructs to suppress the expression of the wild-type or mutant SOD1 mRNA.

In a first aspect, the disclosure features an inhibitory nucleic acid comprising a guide strand (and, optionally, a passenger strand having complementarity to the guide strand). In some embodiments, the guide strand has complementarity sufficient to hybridize to a region within a SOD1 mRNA transcript having the nucleic acid sequence of any one of SEQ ID NOs: 33-48. In some embodiments, the guide strand has at least 70% complementarity to a segment of 15, 16, 17, 18, 19, 20, 21, or more contiguous nucleotides within the region of the SOD1 mRNA transcript having the nucleic acid sequence of any one of SEQ ID NOs: 33-48.

In some embodiments, the guide strand has at least 75% complementarity to a segment of 15, 16, 17, 18, 19, 20, or 21 contiguous nucleotides within the region of the SOD1 mRNA transcript having the nucleic acid sequence of any one of SEQ ID NOs: 33-48. In some embodiments, the guide strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to a segment of 15, 16, 17, 18, 19, 20, 21, or more contiguous nucleotides within the region of the SOD1 mRNA transcript having the nucleic acid sequence of any one of SEQ ID NOs: 33-48.

In some embodiments, the guide strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to a segment of 15 contiguous nucleotides within the region of the SOD1 mRNA transcript having the nucleic acid sequence of any one of SEQ ID NOs: 33-48.

In some embodiments, the guide strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to a segment of 16 contiguous nucleotides within the region of the SOD1 mRNA transcript having the nucleic acid sequence of any one of SEQ ID NOs: 33-48.

In some embodiments, the guide strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to a segment of 17 contiguous nucleotides within the region of the SOD1 mRNA transcript having the nucleic acid sequence of any one of SEQ ID NOs: 33-48.

In some embodiments, the guide strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to a segment of 18 contiguous nucleotides within the region of the SOD1 mRNA transcript having the nucleic acid sequence of any one of SEQ ID NOs: 33-48.

In some embodiments, the guide strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to a segment of 19 contiguous nucleotides within the region of the SOD1 mRNA transcript having the nucleic acid sequence of any one of SEQ ID NOs: 33-48.

In some embodiments, the guide strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to a segment of 20 contiguous nucleotides within the region of the SOD1 mRNA transcript having the nucleic acid sequence of any one of SEQ ID NOs: 33-48.

In some embodiments, the guide strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to a segment of 21 contiguous nucleotides within the region of the SOD1 mRNA transcript having the nucleic acid sequence of any one of SEQ ID NOs: 33-48.

In some embodiments, the guide strand comprises at least 10, at least 11, at least 12, at least 13, at least 14, at least 15, at least 16, at least 17, at least 18, at least 19, at least 20, or at least 21 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises at least 10 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises at least 11 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises at least 12 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises at least 13 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises at least 14 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises at least 15 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises at least 16 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises at least 17 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises at least 18 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises at least 19 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises at least 20 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises 21 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises from 10 to 21 contiguous nucleotides that are fully complementary to a contiguous polynucleotide of equal length within the region of the SOD1 mRNA transcript having the nucleic acid sequence of any one of SEQ ID NOs: 33-48. In some embodiments, the guide strand comprises from 12 to 21 contiguous nucleotides that are fully complementary to a contiguous polynucleotide of equal length within the region of the SOD1 mRNA transcript having the nucleic acid sequence of any one of SEQ ID NOs: 33-48. In some embodiments, the guide strand comprises from 15 to 21 contiguous nucleotides that are fully complementary to a contiguous polynucleotide of equal length within the region of the SOD1 mRNA transcript having the nucleic acid sequence of any one of SEQ ID NOs: 33-48. In some embodiments, the guide strand comprises from 18 to 21 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises 19, 20, or 21 contiguous nucleotides that are fully complementary to a contiguous polynucleotide of equal length within the region of the SOD1 mRNA transcript having the nucleic acid sequence of any one of SEQ ID NOs: 33-48.

In some embodiments, the guide strand comprises 9 or fewer nucleotide mismatches relative to a segment of 15, 16, 17, 18, 19, 20, or 21 contiguous nucleotides within the region of the SOD1 mRNA transcript having the nucleic acid sequence of any one of SEQ ID NOs: 33-48, optionally wherein the guide strand comprises 8 or fewer, 7 or fewer, 6 or fewer, 5 or fewer, 4 or fewer, 3 or fewer, 2 or fewer, or only 1 mismatch relative to the region of the SOD1 mRNA transcript having the nucleic acid sequence of any one of SEQ ID NOs: 33-48.

In some embodiments, the region of the SOD1 mRNA transcript has the nucleic acid sequence of any one of SEQ ID NOs: 33, 38, 40, 41, 42 and 45. In some embodiments, the region of the SOD1 mRNA transcript has the nucleic acid sequence of any one of SEQ ID NOs: 33 and 41. In some embodiments, the region of the SOD1 mRNA transcript has the nucleic acid sequence of any one of SEQ ID NOs: 33, 41 and 42. In some embodiments, the region of the SOD1 mRNA transcript has the nucleic acid sequence of any one of SEQ ID NOs: 40, 41, 42 and 45. In some embodiments, the region of the SOD1 mRNA transcript has the nucleic acid sequence of any one of SEQ ID NOs: 41, 42, and 45.

In some embodiments, the guide strand has a nucleic acid sequence that is at least 85% identical to the nucleic acid sequence of any one of SEQ ID NOs: 1-16 (e.g., at least 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 1-16). In some embodiments, the guide strand has a nucleic acid sequence that is at least 90% identical to the nucleic acid sequence of any one of SEQ ID NOs: 1-16 (e.g., at least 90%, 91%, 92%, 93%, 94%, 95%, 96%, 97%, 98%, 99%, or 100% identical to the nucleic acid sequence of any one of SEQ ID NOs: 1-16). In some embodiments, the guide strand has a nucleic acid sequence that is at least 95% identical to the nucleic acid sequence of SEQ ID NOs: 1-16 (e.g., at least 95%, 96%, 97%, 98%, 99%, or 100% identical to the nucleic acid sequence of any one of SEQ ID NOs: 1-16). In some embodiments, the guide strand has the nucleic acid sequence of any one of SEQ ID NOs: 1-16. In some embodiments, the nucleic acid sequence is any one of SEQ ID NOs: 1, 6, 8, 9, 10 and 13. In some embodiments, the nucleic acid sequence is any one of SEQ ID NOs: 1 and 9. In some embodiments, the nucleic acid sequence is any one of SEQ ID NOs: 1, 9 and 10. In some embodiments, the nucleic acid sequence is any one of SEQ ID NOs: 8, 9, 10 and 13. In some embodiments, the nucleic acid sequence is any one of SEQ ID NOs: 9, 10 and 13.

In some embodiments, the inhibitory nucleic acid comprises a hairpin having a nucleic acid sequence that is at least 85% identical to the nucleic acid sequence of any one of SEQ ID NOs: 17-32 (e.g., at least 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 17-32). In some embodiments, the inhibitory nucleic acid comprises a hairpin having a nucleic acid sequence that is at least 90% identical to the nucleic acid sequence of any one of SEQ ID NOs: 17-32 (e.g., at least 90%, 91%, 92%, 93%, 94%, 95%, 96%, 97%, 98%, 99%, or 100% identical to the nucleic acid sequence of any one of SEQ ID NOs: 17-32). In some embodiments, the hairpin has a nucleic acid sequence that is at least 95% identical to the nucleic acid sequence of any one of SEQ ID NOs: 17-32 (e.g., at least 95%, 96%, 97%, 98%, 99%, or 100% identical to the nucleic acid sequence of any one of SEQ ID NOs: 17-32). In some embodiments, the hairpin has the nucleic acid sequence of any one of SEQ ID NOs: 17-32. In some embodiments, the nucleic acid sequence is any one of SEQ ID NOs: 17, 22, 24, 25, 26 and 29. In some embodiments, the nucleic acid sequence is any one of SEQ ID NOs: 17 and 25. In some embodiments, the nucleic acid sequence is any one of SEQ ID NOs: 17, 25 and 26. In some embodiments, the nucleic acid sequence is any one of SEQ ID NOs: 24, 25, 26 and 29. In some embodiments, the nucleic acid sequence is any one of SEQ ID NOs: 25, 26 and 29.

In some embodiments, the inhibitory nucleic acid is an interfering RNA molecule. In some embodiments, the interfering RNA molecule is a microRNA (miRNA), short hairpin RNA (shRNA), or short interfering RNA (siRNA). In some embodiments, the inhibitory nucleic acid is a miRNA.

In another aspect, the disclosure features a viral vector comprising a transgene encoding the inhibitory nucleic acid of any of the above aspects or embodiments of the disclosure. In some embodiments, the viral vector comprises a plurality of the transgenes (e.g., 2, 3, 4, 5, or more of the transgenes).

In some embodiments, the viral vector is selected from the group consisting of adeno-associated virus (AAV), adenovirus, lentivirus, retrovirus, poxvirus, baculovirus, herpes simplex virus, vaccinia virus, and a synthetic virus. In some embodiments, the viral vector is an AAV.

In some embodiments, the AAV is an AAV1, AAV2, AAV3, AAV4, AAV5, AAV6, AAV7, AAV8, AAV9, AAVrh10, or AAVrh74 serotype. In some embodiments, the viral vector is a pseudotyped AAV. In some embodiments, the pseudotyped AAV has the ITRs of one AAV serotype (e.g., AAV2) and the VP1, VP2, and/or VP3 capsid proteins from a different AAV serotype (e.g., AAV1, AAV2, AAV3, AAV4, AAV5, AAV6, AAV7, AAV8, AAV9, AAVrh10, or AAVrh74). In some embodiments, the pseudotyped AAV is AAV2/9. In some embodiments, the pseudotyped AAV is AAV2/8. In some embodiments, the AAV comprises a recombinant capsid protein.

In some embodiments, the AAV comprises a capsid disclosed, e.g., in WO 2017/218842, the disclosure of which is incorporated herein by reference. In some embodiments, the AAV comprises a capsid protein disclosed in Lin et al. Mol Brain 13:138 (2020), the disclosure of which is incorporated herein by reference. In some embodiments, the AAV comprises an AAV2-retro or an AAV9-retro capsid protein. In some embodiments, the AAV comprises a capsid protein that is conjugated to a ligand or an aptamer.

In some embodiments, the synthetic virus is chimeric virus, mosaic virus, or pseudotyped virus, and/or comprises a foreign protein, synthetic polymer, nanoparticle, or small molecule.

In a further aspect, the disclosure features a pharmaceutical composition comprising the inhibitory nucleic acid or the viral vector of any of the above aspects or embodiments of the disclosure, along with a pharmaceutically acceptable excipient, carrier, or diluent.

In another aspect, the disclosure features a method of treating a neurological disorder in a subject in need thereof by administering to the subject a therapeutically effective amount of the inhibitory nucleic acid, viral vector, or pharmaceutical composition of any of the above aspects or embodiments of the disclosure.

In some embodiments, the neurological disorder is a neurodegenerative disorder. In some embodiments, the neurological disorder is caused by, or associated with, expression of a wild-type or mutant form of SOD1. In some embodiments, the neurological disorder is amyotrophic lateral sclerosis (ALS), Huntington's disease, Parkinson's disease, frontotemporal lobar dementia, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy, dementia Parkinsonism ALS complex of guam, Pick's disease, Perry syndrome, cerebral age-related TDP-43 with sclerosis, hippocampal sclerosis, or Alzheimer's disease. In some embodiments, the neurological disorder is ALS.

In another aspect, the disclosure features a method of treating a cell proliferation disorder in a subject in need thereof by administering to the subject a therapeutically effective amount of the inhibitory nucleic acid, viral vector, or pharmaceutical composition of any of the above aspects or embodiments of the disclosure.

In some embodiments, the cell proliferation disorder is cancer. In some embodiments, the cell proliferation disorder is leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, throat cancer, acute lymphoblastic leukemia, acute myeloid leukemia, chronic lymphocytic leukemia, chronic myelogenous leukemia, adrenocortical carcinoma, acquired immune deficiency syndrome-related lymphoma, primary central nervous system lymphoma, anal cancer, appendix cancer, astrocytoma, atypical teratoid rhabdoid tumor, basal cell carcinoma, bile duct cancer, extrahepatic cancer, Ewing's sarcoma, osteosarcoma, malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ, endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors, testicular germ cell tumor, gestational trophoblastic disease, glioma, childhood brain stem glioma, hairy cell leukemia, hepatocellular cancer, langerhans cell histiocytosis, Hodgkin's lymphoma, hypopharyngeal cancer, islet cell tumors, pancreatic neuroendocrine tumors, Wilms' tumor, childhood kidney tumors, nephroblastoma, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma, plasma cell neoplasms, plasmacytoma, myelodysplastic syndromes, nasopharyngeal cancer, neuroblastoma, non-Hodgkin's lymphoma, non-small cell lung cancer, epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, Kaposi sarcoma, Sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, or Waldenstrom's macroglobulinemia.

In some embodiments, the subject is a mammal (e.g., a human).

In some embodiments, the inhibitory nucleic acid, viral vector, or pharmaceutical composition of any of the above aspects or embodiments of the disclosure is administered to the subject by a route selected from intrathalamic, intrathecal, subpial, intraparenchymal, intrastriatal, intracranial, intracisternal, intracerebral, intracerebroventricular, intraocular (e.g., intravitreal), intraventricular, intralumbar, intravenous, intramuscular, subcutaneous, intraperitoneal, intradermal, transdermal, parenteral, intranasal, percutaneous, intratracheal, intraarterial, intravascular, and oral administration, inhalation, perfusion, lavage, or any combination thereof.

In another aspect, the disclosure features a kit comprising the inhibitory nucleic acid, viral vector, or pharmaceutical composition of any of the above aspects or embodiments of the disclosure. The kit may further include a package insert instructing the use of the kit to administer a therapeutically effective amount of the inhibitory nucleic acid, viral vector, or pharmaceutical composition to a subject (e.g., a mammal, such as a human, diagnosed as having a neurological disease described herein).

### BRIEF DESCRIPTION OF THE DRAWINGS

FIG. 1A is a bar graph showing the knockdown efficacy of a series of SOD1-specific miRNA constructs. Data were obtained from a dual luciferase reporter (DLR) assay performed in HEK cells after standard plasmid transfection. Data represent 3 independent experiments with biological replicates. FIG. 1A abbreviations: SD, Superoxide dismutase 1.

FIG. 1B is a bar graph showing the knockdown efficacy of a series of SOD1-specific miRNA constructs. Data were obtained from a probe-based qPCR assay performed in HEK cells after standard plasmid transfection. Data represent 3 independent experiments with biological replicates. FIG. 1B abbreviations: SD, Superoxide dismutase 1; KD, Knockdown.

FIG. 2A is a bar graph showing the knockdown efficacy of a series of SOD1-specific miRNA constructs. Data were obtained from a dual luciferase reporter (DLR) assay performed in HEK cells after viral plasmid transfection. Control (CONT) is a non-targeting miRNA. Data represent 3 independent experiments with biological replicates. FIG. 2A abbreviations: SD, Superoxide dismutase 1.

FIG. 2B is a bar graph showing the knockdown efficacy of a series of SOD1-specific miRNA constructs. Data were obtained from a probe-based qPCR assay performed in HEK cells after viral plasmid transfection. Control (CONT) is a non-targeting miRNA. Data represent 3-5 independent experiments with biological replicates. FIG. 2B abbreviations: SD, Superoxide dismutase 1; qPCR, quantitative polymerase chain reaction.

FIG. 3A is a bar graph showing the knockdown efficacy of a series of SOD1-specific miRNA constructs. Data were obtained from a probe-based qPCR assay performed in HEK293 cells after viral transduction. Control (neg) is a non-targeting miRNA. Data represent 3 independent experiments with biological replicates. FIG. 3A abbreviations: SOD1, Superoxide dismutase 1; HEK, Human embryonic kidney.

FIG. 3B is a bar graph showing the knockdown efficacy of a series of SOD1-specific miRNA constructs. Data were obtained from a probe-based qPCR assay performed in SH-SY5Y cells after viral transduction. Control (neg) is a non-targeting miRNA. Data represent 3 independent experiments with biological replicates. FIG. 3B abbreviations: SOD1, Superoxide dismutase 1.

### DEFINITIONS

As used herein, the term “about” refers to a value that is within 10% above or below the value being described. For example, the phrase “about 100 nucleic acid residues” refers to a value of from 90 to 110 nucleic acid residues.

As used herein, the term “anneal” refers to the formation of a stable duplex of nucleic acids by way of hybridization mediated by inter-strand hydrogen bonding, for example, according to Watson-Crick base pairing. The nucleic acids of the duplex may be, for example, at least 50% complementary to one another (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% complementary to one another. The “stable duplex” formed upon the annealing of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties of naturally-occurring amino acids

| | | | | Electrostatic | |
|---|---|---|---|---|---|
| | | | Side- | character at | |
| | 3 Letter | 1 Letter | chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the “length” of a nucleic acid refers to the linear size of the nucleic acid as assessed by measuring the quantity of nucleotides from the 5′ to the 3′ end of the nucleic acid. Exemplary molecular biology techniques that may be used to determine the length of a nucleic acid of interest are known in the art.

As used herein, the term “operably linked” refers to a first molecule (e.g., a first nucleic acid) joined to a second molecule (e.g., a second nucleic acid), wherein the molecules are so arranged that the first molecule affects the function of the second molecule. The two molecules may or may not be part of a single contiguous molecule and may or may not be adjacent to one another. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, one segment of a nucleic acid molecule is considered to “overlap with” another segment of the same nucleic acid molecule if the two segments share one or more constituent nucleotides. For example, two segments of the same nucleic acid molecule are considered to “overlap with” one another if the two segments share 1, 2, 3, 4, 5, 6, 7, 8, 9, 10, 15, 20, 25, 30, 35, 40, 45, 50, 100, or more, constituent nucleotides. The two segments are not considered to “overlap with” one another if the two segments have zero constituent nucleotides in common.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

$$100 multiplied by ( the fraction X / Y )$$

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, such as a nucleic acid or vector described herein, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions, and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “wild-type” or “non-mutant” form of a gene refers to a nucleic acid that encodes a protein associated with normal or non-pathogenic activity (e.g., a protein lacking a mutation that results in higher risk of developing, onset, or progression of a neurodegenerative disease).

As used herein, the term “mutation” refers to any change in the structure of a gene, e.g., gene sequence, resulting in an altered form of the gene, which may be passed onto subsequent generations (hereditary mutation) or not (somatic mutation). Gene mutations include the substitution, insertion, or deletion of a single base in DNA or the substitution, insertion, deletion, or rearrangement of multiple bases or larger sections of genes or chromosomes, including repeat expansions.

As used herein, the term “Superoxide dismutase 1” or “SOD1” refers to a protein encoded by the SOD1 gene. SOD1 gene or transcript may refer to normal alleles of SOD1, or mutated alleles with mutations such as D90A, A4V, H46R and G93A, among others. In some embodiments, SOD1 refers to mammalian SOD1, including human SOD1. Exemplary SOD1 proteins that may be targeted using the compositions and methods of the disclosure include a protein having the amino acid sequence represented by NCBI ID NP_000445.1, as well as naturally occurring variants thereof. In some embodiments, an exemplary SOD1 gene has the nucleic acid sequence of NCBI ID NC_000021.9:31659693-31668931, or a naturally occurring variant thereof. Exemplary SOD1 mRNA transcripts include those having the nucleic acid sequence of NCBI ID NM_000454.5, as well as naturally occurring variants thereof.

As used herein, the term “inhibitory nucleic acid” refers to a nucleic acid that comprises a guide strand sequence that hybridizes to at least a portion of a target nucleic acid, e.g., SOD1 RNA, mRNA, pre-mRNA, or mature mRNA, and inhibits its expression or activity. An inhibitory nucleic acid may target a protein coding region (e.g., exon) or non-coding region (e.g., 5′UTR, 3′UTR, intron, etc.) of a target nucleic acid. In some embodiments, an inhibitory nucleic acid is a single stranded or double stranded molecule. An inhibitory nucleic acid may further comprise a passenger strand sequence on a separate strand (e.g., double stranded duplex) or in the same strand (e.g., single stranded, self-annealing duplex structure). In some embodiments, an inhibitory nucleic acid is an interfering RNA molecule, such as short interfering RNA (siRNA), short hairpin RNA (shRNA), microRNA (miRNA), or double-stranded RNA (dsRNA).

As used herein, the term “interfering RNA” refers to an RNA, such as an siRNA, miRNA, or shRNA that suppresses the expression of a target RNA transcript by way of (i) annealing to the target RNA transcript, thereby forming a nucleic acid duplex; and (ii) promoting the nuclease-mediated degradation of the RNA transcript and/or (iii) slowing, inhibiting, or preventing the translation of the RNA transcript, such as by sterically precluding the formation of a functional ribosome-RNA transcript complex or otherwise attenuating formation of a functional protein product from the target RNA transcript. Interfering RNAs as described herein may be provided to a patient, such as a human patient having myotonic dystrophy, in the form of, for example, a single- or double-stranded oligonucleotide, or in the form of a vector (e.g., a viral vector, such as an adeno-associated viral vector described herein) containing a transgene encoding the interfering RNA. Exemplary interfering RNA platforms are described, for example, in Lam et al., Molecular Therapy—Nucleic Acids 4:e252 (2015); Rao et al., Advanced Drug Delivery Reviews 61:746-769 (2009); and Borel et al., Molecular Therapy 22:692-701 (2014), the disclosures of each of which are incorporated herein by reference in their entirety.

As used herein, a “microRNA” or “miRNA” refers to a small non-coding RNA molecule capable of mediating silencing of a target gene by cleavage of the target mRNA, translational repression of the target mRNA, target mRNA degradation, or a combination thereof. Typically, miRNA is transcribed as a hairpin or stem-loop (e.g., having a self-complementary, single-stranded backbone) duplex structure, referred to as a primary miRNA (pri-miRNA), which is enzymatically processed (e.g., by Drosha, DGCR8, Pasha, etc.) into a pre-miRNA. Pre-miRNA is exported into the cytoplasm, where it is enzymatically processed by Dicer to produce a miRNA duplex with the passenger strand and then a single-stranded mature miRNA molecule, which is subsequently loaded into the RNA-induced silencing complex (RISC). Reference to a miRNA may include synthetic or artificial miRNAs.

As used herein, a “synthetic miRNA” or “artificial miRNA” or “amiRNA” refers to an endogenous, modified, or synthetic pri-miRNA or pre-miRNA (e.g., miRNA backbone or scaffold) in which the endogenous miRNA guide sequence and passenger sequence within the stem sequence have been replaced with a miRNA guide sequence and a miRNA passenger sequence that direct highly efficient RNA silencing of the targeted gene (see, e.g., Eamens et al. (2014), Methods Mol. Biol. 1062:211-224). In some embodiments, the nature of the complementarity of the guide and passenger sequences (e.g., number of bases, position of mismatches, types of bulges, etc.) can be similar or different from the nature of complementarity of the guide and passenger sequences in the endogenous miRNA backbone upon which the synthetic miRNA is constructed.

As used herein, the term “microRNA backbone,” “miR backbone,” “microRNA scaffold,” or “miR scaffold” refers to a pri-miRNA or pre-miRNA scaffold, with the stem sequence replaced by a miRNA of interest, and is capable of producing a functional, mature miRNA that directs RNA silencing at the gene targeted by the miRNA of interest. A miR backbone comprises a 5′ flanking region (also referred to 5′ miR context, >9 nucleotides), a stem region comprising the miRNA duplex (guide strand sequence and passenger strand sequence) and basal stem (5′ and 3′, each about 4-13 nucleotides), at least one loop motif region including the terminal loop (210 nucleotides for terminal loop), a 3′ flanking region (also referred to 3′ miR context, >9 nucleotides), and optionally one or more bulges in the stem. A miR backbone may be derived completely or partially from a wild type miRNA scaffold or be a completely artificial sequence.

As used herein, the term “antisense strand sequence” or “guide strand sequence” of an inhibitory nucleic acid refers to a sequence that is substantially complementary (e.g., at least 45%, 50%, 55%, 60%, 65%, 70%, 75%, 80%, 85%, 90%, 95%, or 100% complementary) to a region of about 10-50 nucleotides (e.g., about 15-30, 16-25, 18-23, or 19-22 nucleotides) of the mRNA of the gene targeted for silencing. The antisense sequence is sufficiently complementary to the target mRNA sequence to direct target-specific silencing, e.g., to trigger the destruction of the target mRNA by the RNAi machinery or process. In some embodiments, the antisense sequence or guide strand sequence refers to the mature sequence remaining following cleavage by Dicer.

As used herein, the term “sense sequence” or “passenger strand sequence” of an inhibitory nucleic acid refers to a sequence that is homologous to the target mRNA and partially or completely complementary to the antisense strand sequence or guide strand sequence of an inhibitory nucleic acid. The antisense strand sequence and sense strand sequence of an inhibitory nucleic acid are hybridized to form a duplex structure (e.g., forming a double-stranded duplex or single-stranded self-annealing duplex structure). In some embodiments, the sense sequence or passenger strand sequence refers to the mature sequence remaining following cleavage by Dicer.

As used herein, a “duplex,” when used in reference to an inhibitory nucleic acid, refers to two nucleic acid strands (e.g., a guide strand and passenger strand) hybridizing together to form a duplex structure. A duplex may be formed by two separate nucleic acid strands or by a single nucleic acid strand having a region of self-complementarity (e.g., hairpin or stem-loop).

As used herein, “expression construct” refers to any type of genetic construct containing a nucleic acid (e.g., transgene) in which part or all the nucleic acid encoding sequence is capable of being transcribed. In some embodiments, expression includes transcription of the nucleic acid, for example, to generate a biologically active polypeptide product or inhibitory RNA (e.g., siRNA, shRNA, miRNA) from a transcribed gene. In some embodiments, the transgene is operably linked to expression control sequences.

As used herein, the term “transgene” refers to an exogenous nucleic acid that has been transferred naturally or by genetic engineering means into another cell and is capable of being transcribed, and optionally translated.

As used herein, the term “gene expression” refers to the process by which a nucleic acid is transcribed from a nucleic acid molecule, and often, translated into a peptide or protein. The process can include transcription, post-transcriptional control, post-transcriptional modification, translation, post-translational control, post translational modification, or any combination thereof. Reference to a measurement of “gene expression” may refer to measurement of the product of transcription (e.g., RNA or mRNA), the product of translation (e.g., peptides or proteins).

As used herein, the term “inhibit expression of a gene” means to reduce, down-regulate, suppress, block, lower, or stop expression of the gene. The expression product of a gene can be an RNA molecule transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

As used herein, “neurological disease” or “neurological disorder” refers to diseases or disorders that affect nerves in the brain, spinal cord, and the rest of the human body. A neurological disorder can be caused by electrical, structural, or biochemical abnormalities in neurons, among other causes. As used herein, neurological disorders include neurodegenerative disorders. As used herein, “neurodegenerative disease” or “neurodegenerative disorder” refers to diseases or disorders that exhibit neural cell death as a pathological state. A neurodegenerative disease may exhibit chronic neurodegeneration, e.g., slow, progressive neural cell death over a period of several years, or acute neurodegeneration, e.g., sudden onset or neural cell death. As used herein, the term “neurodegenerative disorder” refers to disorders, diseases or conditions that are caused by the deterioration of cell and tissue components of the nervous system. Some non-limiting examples of neurodegenerative disorders include stroke, Alzheimer's disease, Parkinson's disease, Huntington's disease, Peri ventricular leukomalacia (PVL), amyotrophic lateral sclerosis (ALS, “Lou Gehrig's disease”), ALS-Parkinson's-Dementia complex of Guam, Friedrich's Ataxia, Wilson's disease, multiple sclerosis, cerebral palsy, progressive supranuclear palsy (Steel-Richardson syndrome), bulbar and pseudobulbar palsy, diabetic retinopathy, multi-infarct dementia, macular degeneration, Pick's disease, diffuse Lewy body disease, prion diseases such as Creutzfeldt-Jakob, Gerstmann-Straussler-Scheinker disease, Kuru and fatal familial insomnia, primary lateral sclerosis, degenerative ataxias, Machado-Joseph disease/spinocerebellar ataxia type 3 and olivopontocerebellar degenerations, spinal and spinobulbar muscular atrophy (Kennedy's disease), familial spastic paraplegia, Wohlfart-Kugelberg-Welander disease, Tay-Sach's disease, multisystem degeneration (Shy-Drager syndrome), Gilles De La Tourette's disease, familial dysautonomia (Riley-Day syndrome), Kugelberg-Weiander disease, subacute sclerosing panencephalitis, Werdnig-Hoffmann disease, synucleinopathies (including multiple system atrophy), Sandhoff disease, cortical basal degeneration, spastic paraparesis, primary progressive aphasia, progressive multifocal leukoencephalopathy, striatonigral degeneration, familial spastic disease, chronic epileptic conditions associated with neurodegeneration, Binswanger's disease, and dementia (including all underlying etiologies of dementia). Examples of chronic, neurodegenerative diseases include Alzheimer's disease, Parkinson's disease, Huntington's disease, spinocerebellar ataxia type 2 (SCA2), frontotemporal lobar dementia (FTLD), and amyotrophic lateral schlerosis (ALS). Chronic neurodegenerative diseases include diseases that feature TDP-43 proteinopathy, which is characterized by nucleus to cytoplasmic mislocalization, deposition of ubiquitinated and hyper-phosphorylated TDP-43 into inclusion bodies, protein truncation leading to formation of toxic C-terminal TDP-43 fragments, and protein aggregation. TDP-43 proteinopathy diseases include ALS, FTLD, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, and Alzheimer's disease. Acute neurodegeneration may be caused by ischemia (e.g., stroke, traumatic brain injury), axonal transection by demyelination or trauma (e.g., spinal cord injury or multiple sclerosis). A neurodegenerative disease may exhibit death of mainly one type of neuron or of multiple types of neurons.

As used herein, the term “cell proliferation disorder” refers to diseases or disorders that cause unnatural and uncontrollable cell proliferation or division in any region of the human body and can spread to other parts of the body. Cell proliferation disorders can arise due to genetic defects as well as external factors such as radiation, among other causes. In some embodiments, the cell proliferation disorder is cancer. In some embodiments, the cell proliferation disorder can give rise to a tumor or a cyst. In some embodiments, the tumor or cyst can be benign in nature. In some embodiments, the tumor or cyst can be malignant in nature. Abnormal cells such as tumors or cysts resulting from cell proliferation disorders have the ability to infiltrate and destroy normal body tissue. In some embodiments, the cell proliferation disorder is leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, throat cancer, acute lymphoblastic leukemia, acute myeloid leukemia, chronic lymphocytic leukemia, chronic myelogenous leukemia, adrenocortical carcinoma, acquired immune deficiency syndrome-related lymphoma, primary central nervous system lymphoma, anal cancer, appendix cancer, astrocytoma, atypical teratoid rhabdoid tumor, basal cell carcinoma, bile duct cancer, extrahepatic cancer, Ewing's sarcoma, osteosarcoma, malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ, endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors, testicular germ cell tumor, gestational trophoblastic disease, glioma, childhood brain stem glioma, hairy cell leukemia, hepatocellular cancer, langerhans cell histiocytosis, Hodgkin's lymphoma, hypopharyngeal cancer, islet cell tumors, pancreatic neuroendocrine tumors, Wilms' tumor, childhood kidney tumors, nephroblastoma, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma, plasma cell neoplasms, plasmacytoma, myelodysplastic syndromes, nasopharyngeal cancer, neuroblastoma, non-Hodgkin's lymphoma, non-small cell lung cancer, epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, Kaposi sarcoma, Sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, or Waldenstrom's macroglobulinemia.

As used herein, the term “sample” refers to a specimen (e.g., blood, blood component (e.g., serum or plasma), urine, saliva, amniotic fluid, cerebrospinal fluid, tissue (e.g., placental, or dermal), pancreatic fluid, chorionic villus sample, or cells) isolated from a subject. The subject may be, for example, a patient suffering from a disease described herein, such as a disease associated with expression of SOD1 mutants (example, ALS).

As used herein, the phrases “specifically binds” and “binds” refer to a binding reaction which is determinative of the presence of a particular molecule, such as an RNA transcript, in a heterogeneous population of ions, salts, small molecules, and/or proteins that is recognized, e.g., a mutant SOD1 RNA transcript. A ligand (e.g., an RNA-binding protein described herein) that specifically binds to a species (e.g., an RNA transcript) may bind to the species, e.g., with a K_{D} of less than 1 mM. For example, a ligand that specifically binds to a species may bind to the species with a K_{D} of up to 100 μM (e.g., between 1 pM and 100 μM). A ligand that does not exhibit specific binding to another molecule may exhibit a K_{D} of greater than 1 mM (e.g., 1 μM, 100 μM, 500 μM, 1 mM, or greater) for that particular molecule or ion. A variety of assay formats may be used to determine the affinity of a ligand for a specific protein. For example, solid-phase ELISA assays are routinely used to identify ligands that specifically bind a target protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the terms “subject” and “patient” refer to an organism that receives treatment for a particular disease or condition as described herein (such as a disease associated with expression of an SOD1 mutant, example, ALS). Examples of subjects and patients include mammals, such as humans, receiving treatment for a disease or condition described herein.

As used herein, the term “transcription regulatory element” refers to a nucleic acid that controls, at least in part, the transcription of a gene of interest. Transcription regulatory elements may include promoters, enhancers, and other nucleic acids (e.g., polyadenylation signals) that control or help to control gene transcription. Examples of transcription regulatory elements are described, for example, in Goeddel, Gene Expression Technology: Methods in Enzymology 185 (Academic Press, San Diego, CA, 1990).

As used herein, the terms “treat” or “treatment” refer to therapeutic treatment, in which the object is to prevent or slow down (lessen) an undesired physiological change or disorder, such as the progression of a disease associated with expression of an SOD1 mutant, for example, ALS. In the context of ALS treatment, beneficial or desired clinical results that are indicative of successful treatment include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment of a patient having a neurological disorder (e.g., ALS) may manifest in one or more detectable changes, such as a decrease in the expression of mutant SOD1 RNA transcripts.

As used herein, the term “vector” refers to a nucleic acid, e.g., DNA or RNA, that may function as a vehicle for the delivery of a gene of interest into a cell (e.g., a mammalian cell, such as a human cell), tissue, organ, or organism, such as a patient undergoing treatment for a disease or condition described herein, for purposes of expressing an encoded transgene. Exemplary vectors useful in conjunction with the compositions and methods described herein are plasmids, DNA vectors, RNA vectors, virions, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026, the disclosure of which is incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of transgenes described herein include plasmids that contain regulatory sequences, such as promoter and enhancer regions, which direct gene transcription. Other useful vectors for expression of transgenes contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### DETAILED DESCRIPTION

The compositions and methods described herein are useful for treating disorders associated with expression of wild-type or mutant Superoxide dismutase 1 (SOD1), such as amyotrophic lateral sclerosis (ALS), Parkinson's disease, Huntington's disease, and others. The compositions described herein include inhibitory nucleic acid constructs such as interfering RNA constructs, for example, short interfering RNA (siRNA), short hairpin RNA (shRNA), or microRNA (miRNA) that suppress the expression of wild-type or mutant mRNA transcripts transcribed from wild-type or mutant genes. Without being limited by mechanism, the compositions described herein may ameliorate neuropathology by diminishing the expression of wild-type or mutant mRNA transcripts, thus preventing the expression of the disease phenotypes.

The sections that follow provide a description of exemplary inhibitory nucleic acid of the disclosure, as well as vectors (e.g., viral vectors) encoding the same, and methods of using such inhibitory nucleic acid and vectors for the treatment of neurological diseases.

Superoxide Dismutase 1 (SOD1) and Disorders Associated with SOD1

“SOD1” refers to the Superoxide Dismutase 1 enzyme, which is one of three superoxide dismutases involved in converting harmful superoxide radicals to water. Approximately 10% of all ALS cases are dominantly inherited, and of these ~20% are due to defects in cytosolic superoxide dismutase 1 (SOD1). In addition, SOD1 has been implicated in non-familial (e.g., sporadic) forms of ALS. (Jones, C. T., Brock, D. J. H., Chancellor, A. M., Warlow, C. P., Swingler, R. J. Cu/Zn superoxide dismutase (SOD1) mutations and sporadic amyotrophic lateral sclerosis. Lancet 342: 1050-1051, 1993). Without wishing to be bound by any theory, several lines of investigation have suggested that the mutations in SOD1 do not cause ALS through loss of the dismutase activity of this enzyme. Rather, mutant SOD1 is neurotoxic through multiple alternate mechanisms, many of which entail conformational instability and aberrant binding and aggregation of the mutant protein. While the precise details of the toxicity of mutant SOD1 are not fully defined, it is abundantly clear that reduction of the burden of mutant SOD1 protein in animal models significantly delays death. This has been achieved using both antisense oligonucleotides (ASO) (Smith et al) and siRNA (Maxwell, Pasinelli et al. 2004) (Xia, Zhou et al. 2006) (Wang, Ghosh et al. 2008). These studies illustrate the principle that siRNA-based drugs represent a potentially significant therapeutic advance for the treatment of ALS and many other CNS disorders. In both cases, efficacy was achieved by delivery of high amounts of material over long periods of time; these studies illustrate the point that a major limitation of ASO and siRNA therapies in their present forms is the lack of optimally efficient and non-toxic in vivo delivery systems (Smith, Miller et al. 2006; Wang, Ghosh et al. 2008). ALS is a progressive neurodegenerative disease affecting motor neurons in the central nervous system. Degeneration of the motor neurons results in paralysis and eventual death, usually due to respiratory failure. In a subset of cases, ALS is caused by dominantly transmitted mutations in the gene encoding cytosolic superoxide dismutase (SOD1). Transgenic expression of mutant SOD1 causes ALS in mice.

Aspects of the disclosure relate to inhibitory nucleic acids, such as interfering RNA molecules (e.g., short interfering RNAs (siRNAs), short hairpin RNAs (shRNAs), micro RNAs (miRNAs), including artificial miRNAs), that when administered to a subject reduce the expression or activity of SOD1 in the subject. Accordingly, compositions and methods provided in the present disclosure are useful for the treatment of neurodegenerative diseases, including amyotrophic lateral sclerosis (ALS), Parkinson's disease and Huntington's disease.

### Inhibitory Nucleic Acids

In one aspect, the disclosure provides isolated inhibitory nucleic acids that inhibit expression or activity of SOD1. The inhibitory nucleic acid is a nucleic acid that specifically binds (e.g., hybridizes to) at least a portion of the SOD1 nucleic acid, such as a SOD1 RNA, pre-mRNA, or mRNA, and inhibits its expression or activity. In some embodiments, the inhibitory nucleic acid is complementary to a protein coding region or non-coding region (e.g., 5′UTR, 3′UTR, intron, etc.) of SOD1. In some embodiments, the inhibitory nucleic acid is complementary to a wild type SOD1 nucleic acid or a naturally occurring variant thereof. In some embodiments, the SOD1 allele has mutations such as D90A, A4V, H46R and G93A, among others. In some embodiments, the inhibitory nucleic acid is single stranded or double-stranded. In some embodiments, the inhibitory nucleic acid is an interfering RNA molecule, such as short interfering RNA (siRNA), short hairpin RNA (shRNA), microRNA (miRNA), or double-stranded RNA (dsRNA).

In some embodiments, the inhibitory nucleic acid is a miRNA. A miRNA may be a pri-mRNA, a pre-mRNA, mature miRNA, or artificial miRNA. In some embodiments, a miRNA is comprised of a guide strand and passenger strand. In some embodiments, the guide strand and passenger strand are within the same nucleic acid strand, where the guide strand and passenger strand hybridize together to form a self-annealing duplex structure. MiRNA is initially transcribed as a pri-mRNA, which is processed by nuclear nuclease (e.g., Drosha-DGCR8 complex) into pre-mRNA. A pri-mRNA is a single-stranded molecule having a stem-loop structure. Pre-miRNA is also a single-stranded molecule having a stem-loop structure. The pre-miRNA is transported from the nucleus to the cytoplasm by exportin-5 and further processed by Dicer to produce a mature, double-stranded miRNA duplex comprising a guide strand and a passenger strand. The mature miRNA duplex is then incorporated into the RNA inducing silencing complex (RISC), mediated by TRBP (HIV transactivating response RNA-binding protein). The passenger strand is generally released and cleaved, while the guide strand remains in RISC and binds to the target mRNA and mediates silencing. In some embodiments, a mature miRNA refers to the guide strand of a mature miRNA duplex.

Artificial miRNA refers to an endogenous, modified or synthetic pri-mRNA or pre-mRNA scaffold or backbone capable of producing a functional mature miRNA, where the guide strand sequence and passenger strand sequence of the miRNA duplex within the stem region have been replaced with a guide strand sequence and passenger strand sequence of interest that directs silencing of the target mRNA of interest. Artificial miRNA design is described in Eamens et al. (2014) Methods Mol Biol. 1062:211-24 (incorporated by reference in its entirety). Synthetic miRNA backbones are described in U.S. Patent Publication 2008/0313773 (incorporated by reference in its entirety).

The inhibitory nucleic acid constructs, such as the interfering RNA constructs, described herein may be in any of a variety of forms, such as siRNA, shRNA, or miRNA. The interfering RNAs described herein may additionally be encoded by a vector, such as a viral vector. For example, described herein are adeno-associated viral (AAV) vectors, such as pseudotyped AAV vectors (e.g., AAV2/8 and AAV2/9 vectors) containing transgenes encoding interfering RNA constructs that attenuate the expression of wild-type or mutant RNA transcripts.

The compositions and methods described herein provide, among other benefits, the advantageous feature of being able to selectively suppress the expression of wild-type or pathologic RNA transcripts. Using the compositions and methods described herein, the expression of wild-type RNA transcripts or mutant RNA transcripts can be diminished, while preserving the expression of important healthy RNA transcripts and their encoded protein products.

This advantageous feature is based, in part, on the surprising discovery that inhibitory nucleic acid constructs that anneal to wild-type or mutant RNA targets can be used to suppress the expression of these RNA transcripts. The compositions and methods described herein can thus attenuate the expression of wild-type or pathological RNA transcripts.

The sections that follow provide a description of exemplary inhibitory nucleic acid constructs, such as interfering RNA constructs, that may be used in conjunction with the compositions and methods described herein, as well as a description of vectors encoding such constructs and procedures that may be used to treat diseases associated with expression of wild-type or mutant SOD1.

### Interfering RNA

Using the compositions and methods described herein, a patient having a disease characterized by expression of wild-type or mutant SOD1 may be administered an interfering RNA molecule, a composition containing the same, or a vector encoding the same, so as to suppress the expression of an RNA transcript.

Exemplary interfering RNA molecules that may be used in conjunction with the compositions and methods described herein for the treatment of diseases associated with expression of a SOD1 mutant, such as ALS and others, are siRNA molecules, miRNA molecules, and shRNA molecules, among others. In the case of siRNA molecules, the siRNA may be single stranded or double stranded. miRNA molecules, in contrast, are single-stranded molecules that form a hairpin, thereby adopting a hydrogen-bonded structure reminiscent of a nucleic acid duplex. In either case, the interfering RNA may contain an antisense or “guide” strand that anneals (e.g., by way of complementarity) to the mutant RNA target. The interfering RNA may also contain a “passenger” strand that is complementary to the guide strand and, thus, may have the same nucleic acid sequence as the RNA target.

Exemplary interfering RNA molecules that anneal to SOD1 RNA may be used in conjunction with the compositions and methods described herein for the treatment of diseases associated with expression of wild-type or mutant SOD1 shown in Table 2, below.

TABLE 2
Exemplary inhibitory nucleic acids useful for suppressing SOD1 expression
Exemplary SOD1-directed miRNAs

| | | | Sequence of Region |
|---|---|---|---|
| | Guide Strand | Hairpin Sequence | in SOD1 Transcript |
| miRNA | Sequence | (top strand oligo) | to which miRNA Anneals |
| 1 | CAGGCCUUCAGUC | GAAGGACUGAAUAAAGGCCACUGGAG | AAAGGACUGACUGAAGGCCUG |
| | AGUCCUUU (SEQ | UGAGUAGCAGGUCCAGGCCUUCAGU | (SEQ ID NO: 33) |
| | ID NO: 1) | CAGUCCUUU (SEQ ID NO: 17) | |
| 2 | AUCCAUGCAGGCC | UGAUUGAAGGAUAGCAUGGACAUGAG | UGACUGAAGGCCUGCAUGGAU |
| | UUCAGUCA (SEQ | UGAGUAGCAGGUCAUCCAUGCAGGC | (SEQ ID NO: 34) |
| | ID NO: 2) | CUUCAGUCA (SEQ ID NO: 18) | |
| 3 | AAUCCAUGCAGGC | GGUUGAAGGCAUACAUGGACCUUGA | GACUGAAGGCCUGCAUGGAUU |
| | CUUCAGUC (SEQ | GUGAGUAGCAGGUCAAUCCAUGCAG | (SEQ ID NO: 35) |
| | ID NO: 3) | GCCUUCAGUC (SEQ ID NO: 19) | |
| 4 | AUGGAAUCCAUGC | GGAGGCCUGCAUAGAUUCCACAUGAG | GAAGGCCUGCAUGGAUUCCAU |
| | AGGCCUUC (SEQ | UGAGUAGCAGGUCAUGGAAUCCAUGC | (SEQ ID NO: 36) |
| | ID NO: 4) | AGGCCUUC (SEQ ID NO: 20) | |
| 5 | AACAUGGAAUCCA | GGUUUGCAUGAUAUCCAUGCCUUGA | GGCCUGCAUGGAUUCCAUGUU |
| | UGCAGGCC (SEQ | GUGAGUAGCAGGUCAACAUGGAAUCC | (SEQ ID NO: 37) |
| | ID NO: 5) | AUGCAGGCC (SEQ ID NO: 21) | |
| 6 | GAACAUGGAAUCC | GUUUGCAUGGAUACCAUGUACUCGAG | GCCUGCAUGGAUUCCAUGUUCA |
| | AUGCAGGC (SEQ | UGAGUAGCAGGUCGAACAUGGAAUCC | (SEQ ID NO: 38) |
| | ID NO: 6) | AUGCAGGC (SEQ ID NO: 22) | |
| 7 | AUGAACAUGGAAU | UUGUAUGGAUAUAAUGUUCACAUGAG | CUGCAUGGAUUCCAUGUUCAU |
| | CCAUGCAG (SEQ | UGAGUAGCAGGUCAUGAACAUGGAAU | (SEQ ID NO: 39) |
| | ID NO: 7) | CCAUGCAG (SEQ ID NO: 23) | |
| 8 | UCAUGAACAUGGA | GUAUGGAUUCAUAGUUCAUACGAGAG | GCAUGGAUUCCAUGUUCAUGA |
| | AUCCAUGC (SEQ | UGAGUAGCAGGUCUCAUGAACAUGGA | (SEQ ID NO: 40) |
| | ID NO: 8) | AUCCAUGC (SEQ ID NO: 24) | |
| 9 | AACUCAUGAACAU | UGGGUUCCAUAUACAUGAGACUUGAG | UGGAUUCCAUGUUCAUGAGUU |
| | GGAAUCCA (SEQ | UGAGUAGCAGGUCAACUCAUGAACAU | (SEQ ID NO: 41) |
| | ID NO: 9) | GGAAUCCA (SEQ ID NO: 25) | |
| 10 | UCUUUGUCAGCAG | CGAUGUGACUAUAGACAAAACGAGAG | CAAUGUGACUGCUGACAAAGA |
| | UCACAUUG (SEQ | UGAGUAGCAGGUCUCUUUGUCAGCA | (SEQ ID NO: 42) |
| | ID NO: 10) | GUCACAUUG (SEQ ID NO: 26) | |
| 11 | UCGGCCACACCAU | UGAUAAAGAUAUAGUGGCCACGAGAG | UGACAAAGAUGGUGUGGCCGA |
| | CUUUGUCA (SEQ | UGAGUAGCAGGUCUCGGCCACACCAU | (SEQ ID NO: 43) |
| | ID NO: 11) | CUUUGUCA (SEQ ID NO: 27) | |
| 12 | UCUUCAAUAGACA | GGUCGGUGUGAUAAUUGAAACGAGA | GGCCGAUGUGUCUAUUGAAGA |
| | CAUCGGCC (SEQ | GUGAGUAGCAGGUCUCUUCAAUAGAC | (SEQ ID NO: 44) |
| | ID NO: 12) | ACAUCGGCC (SEQ ID NO: 28) | |
| 13 | UGGACCACCAGUG | UGGUCGUACAAUAGUGGUCACCAGAG | UGGCCGCACACUGGUGGUCCA |
| | UGCGGCCA (SEQ | UGAGUAGCAGGUCUGGACCACCAGU | (SEQ ID NO: 45) |
| | ID NO: 13) | GUGCGGCCA (SEQ ID NO: 29) | |
| 14 | AAUUACACCACAA | CGUUUGGUUUAUAGUGUAAACUUGAG | CGUUUGGCUUGUGGUGUAAUU |
| | GCCAAACG (SEQ | UGAGUAGCAGGUCAAUUACACCACAA | (SEQ ID NO: 46) |
| | ID NO: 14) | GCCAAACG (SEQ ID NO: 30) | |
| 15 | CAGUUUCUCACUA | AGUAUCUGUAAUAAGAAACACUGGAG | AGUACCUGUAGUGAGAAACUG |
| | CAGGUACU (SEQ | UGAGUAGCAGGUCCAGUUUCUCACUA | (SEQ ID NO: 47) |
| | ID NO: 15) | CAGGUACU (SEQ ID NO: 31) | |
| 16 | GGCAAAAUACAGG | UUCGAUGACCAUAAUUUUGACCCGAG | UUCAAUGACCUGUAUUUUGCC |
| | UCAUUGAA (SEQ | UGAGUAGCAGGUCGGCAAAAUACAGG | (SEQ ID NO: 48) |
| | ID NO: 16) | UCAUUGAA (SEQ ID NO: 32) | |

### Methods of Treating Diseases Characterized by Expression of Wild-Type or Mutant SOD1

Using the compositions and methods described herein, a patient experiencing and/or having a disease associated with expression of wild-type or mutant SOD1, such as ALS, Huntington's disease, Parkinson's disease, among others, can be administered an inhibitory nucleic acid construct, such as an interfering RNA construct, for example, short interfering RNA (siRNA), short hairpin RNA (shRNA), microRNA (miRNA), or a vector encoding the same, so as to reduce the expression of wild-type or mutant RNA transcripts.

In another aspect, the present disclosure provides methods for inhibiting the expression or activity of SOD1 in a cell, comprising administering a composition of the present disclosure (e.g., inhibitory nucleic acid, isolated nucleic acid comprising an expression construct encoding an inhibitory nucleic acid, vector, rAAV particle, pharmaceutical composition) to a cell, thereby inhibiting the expression or activity of SOD1 in the cell. In some embodiments, the cell is a CNS cell. In some embodiments, the cell is a non-neuronal cell or neuronal cell of the CNS. In some embodiments, the non-neuronal cell of the CNS is a glial cell, astrocyte, or microglial cell. In some embodiments, the cell is in vitro. In some embodiments, the cell is from a subject having one or more symptoms of a neurodegenerative disease or suspected of having a neurodegenerative disease. In some embodiments, the cell expresses SOD1 having mutations such as D90A, A4V, H46R and G93A, among others.

In another aspect, the present disclosure provides methods for inhibiting the expression or activity of SOD1 in the central nervous system of a subject, comprising administering a composition of the present disclosure (e.g., inhibitory nucleic acid, isolated nucleic acid comprising an expression construct encoding an inhibitory nucleic acid, vector, rAAV particle, pharmaceutical composition) to the subject, thereby inhibiting the expression or activity of SOD1 in the subject.

In another aspect, the present disclosure provides methods for treating a subject having or suspected of having a neurodegenerative disease, comprising administering a composition of the present disclosure (e.g., inhibitory nucleic acid, isolated nucleic acid comprising an expression construct encoding an inhibitory nucleic acid, vector, rAAV particle, pharmaceutical composition) to the subject, thereby treating the subject. As used herein, the term “treat” refers to preventing or delaying onset of neurodegenerative disease (e.g., ALS/FTLD, Alzheimer's disease, Parkinson's disease, etc.); reducing severity of neurodegenerative disease; reducing or preventing development of symptoms characteristic of neurodegenerative disease; preventing worsening of symptoms characteristic of neurodegenerative disease, or any combination thereof.

Neurodegenerative diseases that may be treated in a subject using the compositions of the present disclosure include neurodegenerative diseases where SOD1 is a causative agent (e.g., ALS), as well as neurodegenerative diseases where SOD1 is not the causative agent (e.g., directly causative).

Neurodegenerative diseases associated with mutant SOD1 include ALS, FTLD, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, Perry syndrome, cerebral age-related TDP-43 with sclerosis (CARTS), hippocampal sclerosis, Huntington's disease, Parkinson's disease, and Alzheimer's disease.

In some embodiments, the methods for treatment of the present disclosure reduces, prevents, or slows development or progression of one or more symptom characteristic of a neurodegenerative disease. Examples of symptoms characteristic of neurodegenerative disease include motor dysfunction, cognitive dysfunction, emotional/behavioral dysfunction, or any combination thereof. Paralysis, shaking, unsteadiness, rigidity, twitching, muscle weakness, muscle cramping, muscle stiffness, muscle atrophy, difficulty swallowing, difficulty breathing, speech and language difficulties (e.g., slurred speech), slowness of movement, difficulty with walking, dementia, depression, anxiety, or any combination thereof.

In some embodiments, the methods for treatment of the present disclosure of the present disclosure comprise administration as a monotherapy or in combination with one or more additional therapies for the treatment of the neurodegenerative disease. Combination therapy may mean administration of the compositions of the present disclosure (e.g., inhibitory nucleic acid, isolated nucleic acid comprising an expression construct encoding an inhibitory nucleic acid, vector, rAAV particle, pharmaceutical composition) to the subject concurrently, prior to, subsequent to one or more additional therapies. Concurrent administration of combination therapy may mean that the compositions of the present disclosure (e.g., inhibitory nucleic acid, isolated nucleic acid comprising an expression construct encoding an inhibitory nucleic acid, vector, rAAV particle, pharmaceutical composition) and additional therapy are formulated for administration in the same dosage form or administered in separate dosage forms.

In some embodiments, the one or additional therapies that may be used in combination with the inhibitory nucleic acids of the present disclosure include: inhibitory nucleic acids or antisense oligonucleotides that target neurodegenerative disease related genes or transcripts, gene editing agents (e.g., CRISPR, TALEN, ZFN based systems) that target neurodegenerative related genes, agents that reduce oxidative stress, such as free radical scavengers (e.g., Radicava (edaravone), bromocriptine); antiglutamate agents (e.g., Riluzole, Topiramate, Lamotrigine, Dextromethorphan, Gabapentin and AMPA receptor antagonist (e.g., Talampanel)); Anti-apoptosis agents (e.g., Minocycline, Sodium phenylbutyrate and Arimoclomol); Anti-inflammatory agents (e.g., ganglioside, Celecoxib, Cyclosporine, Nimesulide, Azathioprine, Cyclophosphamide, Plasmapheresis, Glatiramer acetate and thalidomide); Beta-lactam antibiotics (penicillin and its derivatives, ceftriaxone, and cephalosporin); Dopamine agonists (Pramipexole, Dexpramipexole); and neurotrophic factors (e.g., IGF-1, GDNF, BDNF, CTNF, VEGF, Colivelin, Xaliproden, Thyrotrophin-releasing hormone and ADNF).

In some embodiments, a subject treated in any of the methods described herein is a mammal (e.g., mouse, rat), preferably a primate (e.g., monkey, chimpanzee), or human.

In any of the methods of treatment described herein, a composition of the present disclosure (e.g., inhibitory nucleic acid, isolated nucleic acid comprising an expression construct encoding an inhibitory nucleic acid, vector, rAAV particle, pharmaceutical composition) may be administered to the subject by intrathecal, subpial, intraparenchymal, intrastriatal, intracranial, intracisternal, intra-cerebral, intracerebral ventricular, intraocular, intraventricular, intralumbar administration, or any combination thereof.

In some embodiments, a composition of the present disclosure (e.g., inhibitory nucleic acid, isolated nucleic acid comprising an expression construct encoding an inhibitory nucleic acid, vector, rAAV particle, pharmaceutical composition) is directly injected into the CNS of the subject. In some embodiments, direct injection into the CNS is intracerebral injection, intraparenchymal injection, intrathecal injection, intrastriatal injection, subpial injection, or any combination thereof. In some embodiments, direct injection into the CNS is direct injection into the cerebrospinal fluid (CSF) of the subject, optionally wherein the direct injection is intracisternal injection, intraventricular injection, intralumbar injection, or any combination thereof.

### Methods of Treating Cell Proliferation Disorders

Using the compositions and methods described herein, a patient experiencing and/or having a cell proliferation disorder can be administered an inhibitory nucleic acid construct, such as an interfering RNA construct, for example, short interfering RNA (siRNA), short hairpin RNA (shRNA), microRNA (miRNA), or a vector encoding the same, so as to reduce the expression of wild-type or mutant RNA transcripts, such as wild-type or mutant SOD1 transcripts. In some embodiments, the cell proliferation disorder is cancer. SOD1 is an intracellular enzyme that converts superoxide anions to hydrogen peroxide, which is in turn converted to oxygen and water by catalase. Thus, SOD1 plays an important role in counteracting oxidative damage. Resistance to platinum-based therapies such as cisplatin and disease recurrence are major setbacks in cancer treatment. SOD1 can be responsible for conferring oxidative stress resistance against platinum compounds. Silencing or knockdown of SOD1 helps enhance cisplatin-based cytotoxicity such that tumor growth is inhibited. The compositions described herein are useful therapeutics for the treatment of a wide array of cell proliferation disorders and can be administered to a mammalian subject, such as a human, suffering from a cell proliferation disorder, such as cancer.

The compositions described herein can be administered to a mammalian subject (e.g., a human) suffering from cell proliferation disorders in order to improve the condition of the patient. The compositions described herein can be administered to a subject, e.g., via any of the routes of administration described herein. In some embodiments, the compositions described herein can be administered to the subject by a route selected from intrathalamic, intrathecal, subpial, intraparenchymal, intrastriatal, intracranial, intracisternal, intracerebral, intracerebroventricular, intraocular (e.g., intravitreal), intraventricular, intralumbar, intravenous, intramuscular, subcutaneous, intraperitoneal, intradermal, transdermal, parenteral, intranasal, percutaneous, intratracheal, intraarterial, intravascular, and oral administration, inhalation, perfusion, lavage, or any combination thereof. The compositions described herein can also be formulated with excipients, biologically acceptable carriers, and may be optionally conjugated to, admixed with, or co-administered separately (e.g., sequentially) with additional therapeutic agents, such as anti-cancer agents. Cell proliferation disorders that can be treated by the compositions and methods described herein include leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, throat cancer, acute lymphoblastic leukemia, acute myeloid leukemia, chronic lymphocytic leukemia, chronic myelogenous leukemia, adrenocortical carcinoma, acquired immune deficiency syndrome-related lymphoma, primary central nervous system lymphoma, anal cancer, appendix cancer, astrocytoma, atypical teratoid rhabdoid tumor, basal cell carcinoma, bile duct cancer, extrahepatic cancer, Ewing's sarcoma, osteosarcoma, malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ, endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors, testicular germ cell tumor, gestational trophoblastic disease, glioma, childhood brain stem glioma, hairy cell leukemia, hepatocellular cancer, langerhans cell histiocytosis, Hodgkin's lymphoma, hypopharyngeal cancer, islet cell tumors, pancreatic neuroendocrine tumors, Wilms' tumor, childhood kidney tumors, nephroblastoma, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma, plasma cell neoplasms, plasmacytoma, myelodysplastic syndromes, nasopharyngeal cancer, neuroblastoma, non-Hodgkin's lymphoma, non-small cell lung cancer, epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, Kaposi sarcoma, Sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, or Waldenstrom's macroglobulinemia.

### Vectors for Delivery of Inhibitory Nucleic Acid

### Viral Vectors for Inhibitory Nucleic Acid Delivery

Viral genomes provide a rich source of vectors that can be used for the efficient delivery of a gene of interest into the genome of a target cell in a patient (e.g., a mammalian cell, such as a human cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors that may be used in conjunction with the compositions and methods described herein are AAV, retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses that may be used in conjunction with the compositions and methods described herein include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene therapy.

### AAV Vectors for Inhibitory Nucleic Acid Delivery

In some embodiments, inhibitory nucleic acid constructs, such as interfering RNA constructs (for example, short interfering RNA (siRNA), short hairpin RNA (shRNA), or microRNA (miRNA)) described herein are incorporated into recombinant AAV (rAAV) vectors in order to facilitate their introduction into a cell. rAAV vectors useful in the conjunction with the compositions and methods described herein include recombinant nucleic acid constructs that contain (1) a transgene encoding an inhibitory nucleic acid construct, such as an interfering RNA construct described herein (such as an siRNA, shRNA, or miRNA described herein), and (2) one or more nucleic acids that facilitate and expression of the heterologous genes. The viral nucleic acids may include those sequences of AAV that are required in cis for replication and packaging (e.g., functional ITRs) of the DNA into a virion. Such rAAV vectors may also contain marker or reporter genes. Useful rAAV vectors include those having one or more of the naturally-occurring AAV genes deleted in whole or in part, but retain functional flanking ITR sequences. The AAV ITRs may be of any serotype (e.g., derived from serotype 2) suitable for a particular application. Methods for using rAAV vectors are described, for example, in Tal et al., J. Biomed. Sci. 7:279-291 (2000), and Monahan and Samulski, Gene Delivery 7:24-30 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791-801 (2002) and Bowles et al., J. Virol. 77:423-432 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8 and 9. Construction and use of AAV vectors and AAV proteins of different serotypes are described, for example, in Chao et al., Mol. Ther. 2:619-623 (2000); Davidson et al., Proc. Natl. Acad. Sci. USA 97:3428-3432 (2000); Xiao et al., J. Virol. 72:2224-2232 (1998); Halbert et al., J. Virol. 74:1524-1532 (2000); Halbert et al., J. Virol. 75:6615-6624 (2001); and Auricchio et al., Hum. Molec. Genet. 10:3075-3081 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV2) pseudotyped with a capsid gene derived from a serotype other than the given serotype (e.g., AAV1, AAV3, AAV4, AAV5, AAV6, AAV7, AAV8, or AAV9, among others). For example, a representative pseudotyped vector is an AAV2 vector encoding a therapeutic protein pseudotyped with a capsid gene derived from AAV serotype 8 or AAV serotype 9. In some embodiments, the pseudotyped AAV has the ITRs of one AAV serotype (e.g., AAV2) and the VP1, VP2, and/or VP3 capsid proteins from a different AAV serotype (e.g., AAV1, AAV2, AAV3, AAV4, AAV5, AAV6, AAV7, AAV8, AAV9, AAVrh10, or AAVrh74). Techniques involving the construction and use of pseudotyped rAAV virions are known in the art and are described, for example, in Duan et al., J. Virol. 75:7662-7671 (2001); Halbert et al., J. Virol. 74:1524-1532 (2000); Zolotukhin et al., Methods, 28:158-167 (2002); and Auricchio et al., Hum. Molec. Genet., 10:3075-3081 (2001).

In some embodiments, the AAV comprises a capsid disclosed, e.g., in WO 2017/218842, the disclosure of which is incorporated herein by reference. In some embodiments, the AAV comprises a capsid protein disclosed in Lin et al. Mol Brain 13:138 (2020), the disclosure of which is incorporated herein by reference. In some embodiments, the AAV comprises an AAV2-retro or an AAV9-retro capsid protein. In some embodiments, the AAV comprises a capsid protein that is conjugated to a ligand or an aptamer.

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635-45 (2000). Other rAAV virions that can be used in methods of the invention include those capsid hybrids that are generated by molecular breeding of viruses as well as by exon shuffling. See, e.g., Soong et al., Nat. Genet., 25:436-439 (2000) and Kolman and Stemmer, Nat. Biotechnol. 19:423-428 (2001).

### Additional Methods for the Delivery of Inhibitory Nucleic Acids

### Transfection Techniques

Techniques that can be used to introduce a transgene, such as a transgene encoding an inhibitory nucleic acid described herein, into a target cell (e.g., a target cell from or within a human patient suffering from RNA dominance) are known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) and diethylaminoethyl (DEAE)-dextran, the use of which as a transfection agent is described in detail, for example, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, a technique that involves exposing a cell to electromagnetic radiation of a particular wavelength in order to gently permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Incorporation of Genes Encoding Inhibitory Nucleic Acids by Gene Editing

In addition to the above, a variety of tools have been developed that can be used for the incorporation of a transgene, such as a transgene encoding an inhibitory nucleic acid construct, such as an interfering RNA construct (for example, short interfering RNA (siRNA), short hairpin RNA (shRNA), microRNA (miRNA) described herein), into a target cell, and particularly into a human cell. One such method that can be used for incorporating polynucleotides encoding inhibitory nucleic acids into target cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the transgene of interest can be integrated into the genome of a mammalian cell by transposase-catalyzed cleavage of similar excision sites that exist within the nuclear genome of the cell. This allows the transgene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems are the piggybac transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest.

Another tool for the integration of target transgenes into the genome of a target cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can design a CRISPR/Cas system to cleave any target DNA molecule of interest. This technique has been exploited in order to edit eukaryotic genomes (Hwang et al., Nature Biotechnology 31:227 (2013)) and can be used as an efficient means of site-specifically editing target cell genomes in order to cleave DNA prior to the incorporation of a gene encoding a target gene. The use of CRISPR/Cas to modulate gene expression has been described in, for example, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a transgene of interest in a target cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al., Nature Reviews Genetics 11:636 (2010); and in Joung et al., Nature Reviews Molecular Cell Biology 14:49 (2013), the disclosure of each of which are incorporated herein by reference as they pertain to compositions and methods for genome editing.

Additional genome editing techniques that can be used to incorporate polynucleotides encoding target transgenes into the genome of a target cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target transgene into the nuclear DNA of a target cell. These single-chain nucleases have been described extensively in, for example, U.S. Pat. Nos. 8,021,867 and 8,445,251, the disclosures of each of which are incorporated herein by reference as they pertain to compositions and methods for genome editing.

### Methods of Detecting RNA Transcript Expression

The expression level of a wild-type or pathological RNA transcript, such as a wild-type or mutant SOD1 RNA transcript can be ascertained, for example, by a variety of nucleic acid detection techniques. Additionally, or alternatively, RNA transcript expression can be inferred by evaluating the concentration or relative abundance of an encoded protein produced by translation of the RNA transcript. Protein concentrations can also be assessed, for example, using functional assays. Using these techniques, a reduction in the concentration of wild-type or pathological RNA transcripts in response to the compositions and methods described herein can be observed, while monitoring the expression of the encoded protein. The sections that follow describe exemplary techniques that can be used to measure the expression level of a wild-type or pathological RNA transcript and its downstream protein product. RNA transcript expression can be evaluated by a number of methodologies known in the art, including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of RNAs.

### Nucleic Acid Detection

Nucleic acid-based methods for detection of RNA transcript expression include imaging-based techniques (e.g., Northern blotting or Southern blotting), which may be used in conjunction with cells obtained from a patient following administration of, for example, a vector encoding an inhibitory nucleic acid construct (such as an interfering RNA, for example, short interfering RNA (siRNA), short hairpin RNA (shRNA), or microRNA (miRNA) described herein) or a composition containing such an inhibitory nucleic acid construct. Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, NY 11803-2500. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

RNA detection techniques that may be used in conjunction with the compositions and methods described herein to evaluate the expression level of RNA transcripts, such as the SOD1 RNA transcripts, further include microarray sequencing experiments (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms. Additional methods of sequencing known in the art can also be used. For example, transgene expression at the mRNA level may be determined using RNA-Seq (e.g., as described in Mortazavi et al., Nat. Methods 5:621-628 (2008), the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology®). Then, the cDNA is converted into a molecular library for sequencing by addition of sequence adapters for each library (e.g., from Illumina®/Solexa), and the resulting 50-100 nucleotide reads are mapped onto the genome.

RNA expression levels may be determined using microarray-based platforms (e.g., single-nucleotide polymorphism arrays), as microarray technology offers high resolution. Details of various microarray methods can be found in the literature. See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known. Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and comprises arrays fabricated by direct synthesis of oligonucleotides on a glass surface. Other systems may be used as known to one skilled in the art.

Amplification-based assays also can be used to measure the expression level of a particular RNA transcript, such as a wild-type or mutant SOD1 transcript. In such assays, the nucleic acid sequence of the transcript acts as a template in an amplification reaction (for example, PCR, such as qPCR). In a quantitative amplification, the amount of amplification product is proportional to the amount of template in the original sample. Comparison to appropriate controls provides a measure of the expression level of the transcript of interest, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001 (1996), and in Heid et al., Genome Res. 6:986-994 (1996), the disclosures of each of which are incorporated herein by reference in their entirety. Levels of RNA transcript expression as described herein can be determined, for example, by RT-PCR technology. Probes used for PCR may be labeled with a detectable marker, such as, for example, a radioisotope, fluorescent compound, bioluminescent compound, a chemiluminescent compound, metal chelator, or enzyme.

### Protein Detection

Expression of an RNA construct may also be inferred by analyzing expression of the protein encoded by the construct. Protein levels can be assessed using standard detection techniques known in the art. Protein expression assays suitable for use with the compositions and methods described herein include proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including, but not limited to, monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize transgene expression in a cell from a patient (e.g., a human patient) following delivery of the transgene. Any method of MS known in the art may be used to determine, detect, and/or measure a protein or peptide fragment of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample obtained from the patient can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for analyzing complex mixtures. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example, by searching spectra against a database while allowing for specific peptide modifications.

### Pharmaceutical Compositions

The inhibitory nucleic acid constructs, such as interfering RNA constructs (for example, short interfering RNA (siRNA), short hairpin RNA (shRNA), or microRNA (miRNA)), as well as the vectors and compositions encoding or containing such constructs, may be incorporated into a vehicle for administration into a patient, such as a human patient suffering from a disorder, as described herein. Pharmaceutical compositions containing vectors, such as viral vectors, that encode an inhibitory nucleic acid construct described herein can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

Mixtures of the nucleic acids and viral vectors described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for administration by a route selected from intrathalamic, intrathecal, subpial, intraparenchymal, intrastriatal, intracranial, intracisternal, intracerebral, intracerebroventricular, intraocular (e.g., intravitreal), intraventricular, intralumbar, intravenous, intramuscular, subcutaneous, intraperitoneal, intradermal, transdermal, parenteral, intranasal, percutaneous, intratracheal, intraarterial, intravascular, and oral administration, inhalation, perfusion, lavage, or any combination thereof. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations may meet sterility, pyrogenicity, general safety, and purity standards as required by FDA Office of Biologics standards.

A pharmaceutical composition containing, for example, an inhibitory nucleic acid described herein, typically includes a pharmaceutically acceptable diluent or carrier. A pharmaceutical composition may include (e.g., consist of), e.g., a sterile saline solution and a nucleic acid. The sterile saline is typically a pharmaceutical grade saline. A pharmaceutical composition may include (e.g., consist of), e.g., sterile water and a nucleic acid. The sterile water is typically a pharmaceutical grade water. A pharmaceutical composition may include (e.g., consist of), e.g., phosphate-buffered saline (PBS) and a nucleic acid. The sterile PBS is typically a pharmaceutical grade PBS.

In certain embodiments, pharmaceutical compositions include one or more composition or nucleic acid molecule and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, nucleic acid molecules may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions including a nucleic acid molecule encompass any pharmaceutically acceptable salts of the inhibitor, esters of the inhibitor, or salts of such esters. In certain embodiments, pharmaceutical compositions including a nucleic acid molecule, upon administration to a subject (e.g., a human), are capable of providing (directly or indirectly) the biologically active metabolite or residue thereof. Accordingly, for example, the disclosure is also drawn to pharmaceutically acceptable salts of inhibitors, prodrugs, pharmaceutically acceptable salts of such prodrugs, and other bioequivalents. Suitable pharmaceutically acceptable salts include, but are not limited to, sodium and potassium salts. In certain embodiments, prodrugs include one or more conjugate group attached to a nucleic acid molecule, wherein the conjugate group is cleaved by endogenous nucleases within the body.

Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions include a delivery system. Examples of delivery systems include, but are not limited to, liposomes and emulsions. Certain delivery systems are useful for preparing certain pharmaceutical compositions including those including hydrophobic compounds. In certain embodiments, certain organic solvents such as dimethylsulfoxide are used.

In certain embodiments, pharmaceutical compositions include one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions include a co-solvent system. Certain of such co-solvent systems include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intraocular (e.g., intravitreal), intravenous, subcutaneous, intramuscular, intrathecal, intracerebroventricular, etc.). In certain of such embodiments, a pharmaceutical composition includes a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions, or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

### Routes of Administration and Dosing

Viral vectors, such as AAV vectors and others described herein, containing a transgene encoding an inhibitory nucleic acid of the disclosure may be administered to a patient (e.g., a human patient) by a variety of routes of administration. The route of administration may vary, for example, with the onset and severity of disease, and may be selected from, e.g., intrathalamic, intrathecal, subpial, intraparenchymal, intrastriatal, intracranial, intracisternal, intracerebral, intracerebroventricular, intraocular (e.g., intravitreal), intraventricular, intralumbar, intravenous, intramuscular, subcutaneous, intraperitoneal, intradermal, transdermal, parenteral, intranasal, percutaneous, intratracheal, intraarterial, intravascular, and oral administration, inhalation, perfusion, lavage, or any combination thereof. Intravascular administration includes delivery into the vasculature of a patient. In some embodiments, the administration is into a vessel considered to be a vein (intravenous), and in some administration, the administration is into a vessel considered to be an artery (intraarterial). Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Treatment regimens may vary, and often depend on disease severity and the age, weight, and sex of the patient. Treatment may include administration of vectors (e.g., viral vectors) or other agents described herein as useful for the introduction of a transgene into a target cell in various unit doses. Each unit dose will ordinarily contain a predetermined quantity of the therapeutic composition.

### Kits

The compositions and methods described herein can be provided in a kit for use in treating disorders associated with expression of wild-type or mutant superoxide dismutase 1 (SOD1) RNA transcripts (e.g., Parkinson's disease), among others. In some embodiments, the kit may include a pharmaceutical composition of the present disclosure. The kit can include a package insert that instructs a user of the kit, such as a physician of skill in the art, to perform any one of the methods of treatment described herein. The kit may optionally include a syringe or other device for administering the compositions of the present disclosure. In some embodiments, the kit may include one or more additional therapeutic agents.

### EXAMPLES

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. Determining the Knockdown Efficacy of SOD1-Specific miRNA Constructs in HEK Cells Via Dual Luciferase Reporter Assay and Probe-Based qPCR Assay after Standard Plasmid (pMix) Transfection

### Objective

The objective of this study was to evaluate the knockdown efficacy of several SOD1 miRNA candidates in HEK cells via the dual luciferase reporter assay and probe-based qPCR assay. This study was designed to determine the efficiency of the miRNA constructs in silencing SOD1 mRNA.

### Materials and Methods

HEK cells were transfected with standard plasmids (pMix) encoding SOD1-specific miRNA constructs. A dual luciferase reporter assay and a probe-based qPCR assay were then performed to measure the efficacy of the knockdown achieved by each miRNA construct.

The miRNA constructs tested are shown in the following table:

SOD1-directed miRNAs

| | | | Sequence of |
|---|---|---|---|
| | | | Region in SOD1 |
| | Guide Strand | Hairpin Sequence | Transcript to which |
| miRNA | Sequence | (top strand oligo) | miRNA Anneals |
| 1 | CAGGCCUUCAGUC | GAAGGACUGAAUAAAGGCCACUGGAG | AAAGGACUGACUGAAGGCCUG (SEQ |
| | AGUCCUUU (SEQ | UGAGUAGCAGGUCCAGGCCUUCAGU | ID NO: 33) |
| | ID NO: 1) | CAGUCCUUU (SEQ ID NO: 17) | |
| 2 | AUCCAUGCAGGCC | UGAUUGAAGGAUAGCAUGGACAUGAG | UGACUGAAGGCCUGCAUGGAU (SEQ |
| | UUCAGUCA (SEQ | UGAGUAGCAGGUCAUCCAUGCAGGC | ID NO: 34) |
| | ID NO: 2) | CUUCAGUCA (SEQ ID NO: 18) | |
| 3 | AAUCCAUGCAGGC | GGUUGAAGGCAUACAUGGACCUUGA | GACUGAAGGCCUGCAUGGAUU (SEQ |
| | CUUCAGUC (SEQ | GUGAGUAGCAGGUCAAUCCAUGCAG | ID NO: 35) |
| | ID NO: 3) | GCCUUCAGUC (SEQ ID NO: 19) | |
| 4 | AUGGAAUCCAUGC | GGAGGCCUGCAUAGAUUCCACAUGAG | GAAGGCCUGCAUGGAUUCCAU (SEQ |
| | AGGCCUUC (SEQ | UGAGUAGCAGGUCAUGGAAUCCAUGC | ID NO: 36) |
| | ID NO: 4) | AGGCCUUC (SEQ ID NO: 20) | |
| 5 | AACAUGGAAUCCA | GGUUUGCAUGAUAUCCAUGCCUUGA | GGCCUGCAUGGAUUCCAUGUU (SEQ |
| | UGCAGGCC (SEQ | GUGAGUAGCAGGUCAACAUGGAAUCC | ID NO: 37) |
| | ID NO: 5) | AUGCAGGCC (SEQ ID NO: 21) | |
| 6 | GAACAUGGAAUCC | GUUUGCAUGGAUACCAUGUACUCGAG | GCCUGCAUGGAUUCCAUGUUCA (SEQ |
| | AUGCAGGC (SEQ | UGAGUAGCAGGUCGAACAUGGAAUCC | ID NO: 38) |
| | ID NO: 6) | AUGCAGGC (SEQ ID NO: 22) | |
| 7 | AUGAACAUGGAAU | UUGUAUGGAUAUAAUGUUCACAUGAG | CUGCAUGGAUUCCAUGUUCAU (SEQ |
| | CCAUGCAG (SEQ | UGAGUAGCAGGUCAUGAACAUGGAAU | ID NO: 39) |
| | ID NO: 7) | CCAUGCAG (SEQ ID NO: 23) | |
| 8 | UCAUGAACAUGGA | GUAUGGAUUCAUAGUUCAUACGAGAG | GCAUGGAUUCCAUGUUCAUGA (SEQ |
| | AUCCAUGC (SEQ | UGAGUAGCAGGUCUCAUGAACAUGGA | ID NO: 40) |
| | ID NO: 8) | AUCCAUGC (SEQ ID NO: 24) | |
| 9 | AACUCAUGAACAU | UGGGUUCCAUAUACAUGAGACUUGAG | UGGAUUCCAUGUUCAUGAGUU (SEQ |
| | GGAAUCCA (SEQ | UGAGUAGCAGGUCAACUCAUGAACAU | ID NO: 41) |
| | ID NO: 9) | GGAAUCCA (SEQ ID NO: 25) | |
| 10 | UCUUUGUCAGCAG | CGAUGUGACUAUAGACAAAACGAGAG | CAAUGUGACUGCUGACAAAGA (SEQ |
| | UCACAUUG (SEQ | UGAGUAGCAGGUCUCUUUGUCAGCA | ID NO: 42) |
| | ID NO: 10) | GUCACAUUG (SEQ ID NO: 26) | |
| 11 | UCGGCCACACCAU | UGAUAAAGAUAUAGUGGCCACGAGAG | UGACAAAGAUGGUGUGGCCGA (SEQ |
| | CUUUGUCA (SEQ | UGAGUAGCAGGUCUCGGCCACACCAU | ID NO: 43) |
| | ID NO: 11) | CUUUGUCA (SEQ ID NO: 27) | |
| 12 | UCUUCAAUAGACA | GGUCGGUGUGAUAAUUGAAACGAGA | GGCCGAUGUGUCUAUUGAAGA (SEQ |
| | CAUCGGCC (SEQ | GUGAGUAGCAGGUCUCUUCAAUAGAC | ID NO: 44) |
| | ID NO: 12) | ACAUCGGCC (SEQ ID NO: 28) | |
| 13 | UGGACCACCAGUG | UGGUCGUACAAUAGUGGUCACCAGAG | UGGCCGCACACUGGUGGUCCA (SEQ |
| | UGCGGCCA (SEQ | UGAGUAGCAGGUCUGGACCACCAGU | ID NO: 45) |
| | ID NO: 13) | GUGCGGCCA (SEQ ID NO: 29) | |
| 14 | AAUUACACCACAA | CGUUUGGUUUAUAGUGUAAACUUGAG | CGUUUGGCUUGUGGUGUAAUU (SEQ |
| | GCCAAACG (SEQ | UGAGUAGCAGGUCAAUUACACCACAA | ID NO: 46) |
| | ID NO: 14) | GCCAAACG (SEQ ID NO: 30) | |
| 15 | CAGUUUCUCACUA | AGUAUCUGUAAUAAGAAACACUGGAG | AGUACCUGUAGUGAGAAACUG (SEQ |
| | CAGGUACU (SEQ | UGAGUAGCAGGUCCAGUUUCUCACUA | ID NO: 47) |
| | ID NO: 15) | CAGGUACU (SEQ ID NO: 31) | |
| 16 | GGCAAAAUACAGG | UUCGAUGACCAUAAUUUUGACCCGAG | UUCAAUGACCUGUAUUUUGCC (SEQ |
| | UCAUUGAA (SEQ | UGAGUAGCAGGUCGGCAAAAUACAGG | ID NO: 48) |
| | ID NO: 16) | UCAUUGAA (SEQ ID NO: 32) | |

### Results

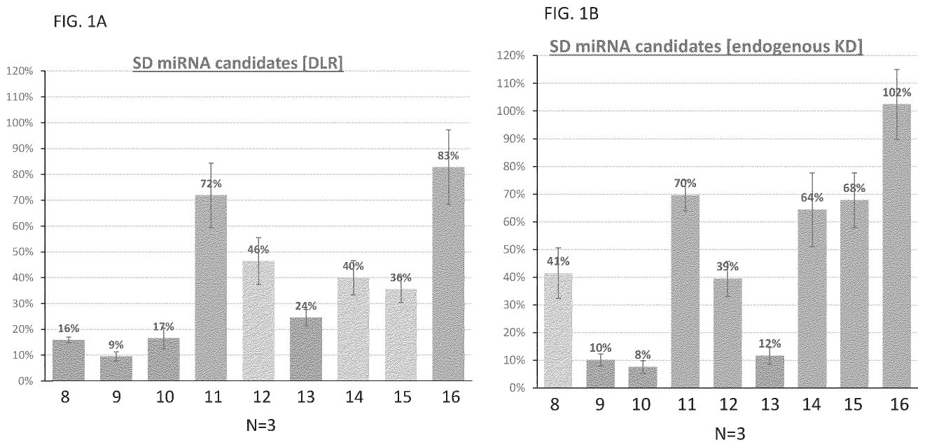

In result, we observed that the SOD1 miRNA candidates were able to silence SOD1 to varying extents (FIG. 1A, 1B), with some miRNA constructs (miRNAs 8, 9,10, and 13) achieving a higher knockdown efficiency compared to other miRNA constructs.

### Example 2. Determining the Knockdown Efficacy of SOD1-Specific miRNA Constructs in HEK Cells Via Dual Luciferase Reporter Assay and Probe-Based qPCR Assay after Viral Plasmid (iMix) Transfection

### Objective

The objective of this study was to evaluate the knockdown efficacy of several SOD1-specific miRNA candidates in HEK cells via a dual luciferase reporter assay and a probe-based qPCR assay after plasmid transfection. This study was designed to determine the efficiency of the miRNA constructs in silencing SOD1 mRNA.

### Materials and Methods

HEK cells were transfected with viral plasmids (iMix) encoding SOD1-specific miRNA constructs. A dual luciferase reporter assay and a probe-based qPCR assay were then performed to measure the efficacy of the knockdown achieved by each miRNA construct in comparison to a negative control, which effectuates no significant silencing.

### Results

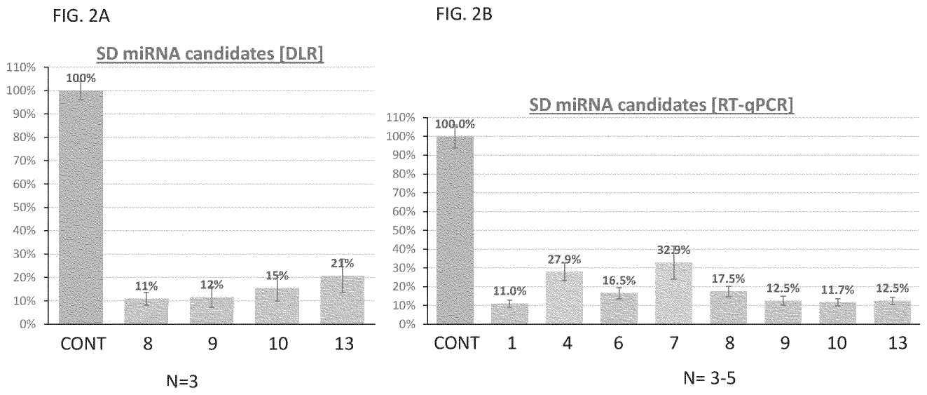

In result, we observed that the SOD1 miRNA candidates were able to silence SOD1 to varying extents (FIG. 2A, 2B), with some miRNA constructs (miRNAs 1, 6, 8, 9, 10, and 13) achieving a higher knockdown efficiency compared to other miRNA constructs. Control (CONT) is a non-targeting miRNA and shows no knockdown.

### Example 3. Determining the Knockdown Efficacy of SOD1-Specific miRNA Constructs in HEK293 Cells and SH-SY5Y Cells Via a Probe-Based qPCR Assay after Viral Transduction

### Objective

The objective of this study was to evaluate the knockdown efficacy of several enodgenous SOD1 miRNA constructs in HEK293 and SH-SY5Y cells via a probe-based qPCR assay after viral transduction. This study was designed to determine the efficiency of SOD1-specific miRNA constructs in silencing the SOD1 mRNA.

### Materials and Methods

HEK293 and SH-SY5Y cells were transduced with AAV vectors (vMix) incorporated with SOD1 miRNA candidates for miRNA delivery and subsequent knockdown of the SOD1 mRNA. A probe-based qPCR assay was then performed to measure the efficacy of the knockdown obtained with the miRNA constructs in comparison with a negative control, which effectuates no significant silencing.

### Results

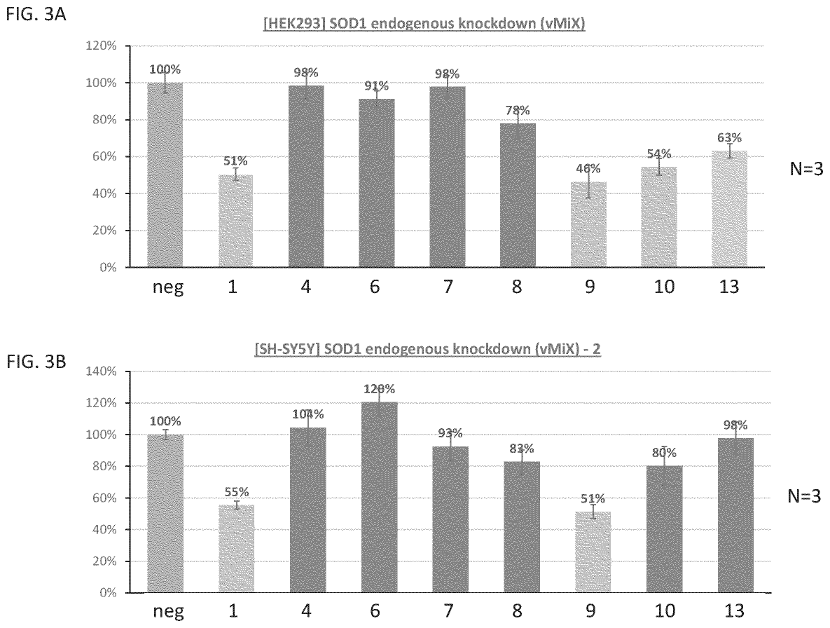

In result, we observed that the SOD1 miRNA candidates were able to silence SOD1 to varying extents in HEK293 cells (FIG. 3A), with some miRNA constructs (miRNAs 1, 9, and 10) achieving a higher knockdown efficiency compared to other miRNA constructs. In result, we observed that the SOD1 miRNA candidates were able to silence SOD1 to varying extents in SH-SY5Y cells (FIG. 3B), with some miRNA constructs (miRNAs 1 and 9) achieving a higher knockdown efficiency compared to other miRNA constructs. Control (neg) is a non-targeting miRNA and shows no knockdown.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. An inhibitory nucleic acid comprising a guide strand and passenger strand having complementarity to the guide strand, wherein the guide strand has complementarity sufficient to hybridize to a region within a superoxide dismutase 1 (SOD1) mRNA transcript having the nucleic acid sequence of any one of SEQ ID NOs: 33-48.

2. The inhibitory nucleic acid of claim 1, wherein the guide strand has at least 70% complementarity to a segment of 15, 16, 17, 18, 19, 20, or 21 contiguous nucleotides within the region of the SOD1 mRNA transcript having the nucleic acid sequence of any one of SEQ ID NOs: 33-48.

3. The inhibitory nucleic acid of claim 2, wherein the guide strand has at least 75% complementarity to a segment of 15, 16, 17, 18, 19, 20, or 21 contiguous nucleotides within the region of the SOD1 mRNA transcript having the nucleic acid sequence of any one of SEQ ID NOs: 33-48, optionally wherein the guide strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to a segment of 15, 16, 17, 18, 19, 20, or 21 contiguous nucleotides within the region of the SOD1 mRNA transcript having the nucleic acid sequence of any one of SEQ ID NOs: 33-48.

4. The inhibitory nucleic acid of any one of claims 1-3, wherein the guide strand comprises at least 10, at least 11, at least 12, at least 13, at least 14, at least 15, at least 16, at least 17, at least 18, at least 19, at least 20, or at least 21 contiguous nucleotides that are fully complementary to a contiguous polynucleotide of equal length within the region of the SOD1 mRNA transcript having the nucleic acid sequence of any one of SEQ ID NOs: 33-48.

5. The inhibitory nucleic acid of claim 4, wherein the guide strand comprises from 10 to 21 contiguous nucleotides that are fully complementary to a contiguous polynucleotide of equal length within the region of the SOD1 mRNA transcript having the nucleic acid sequence of any one of SEQ ID NOs: 33-48.

6. The inhibitory nucleic acid of claim 5, wherein the guide strand comprises from 12 to 21 contiguous nucleotides that are fully complementary to a contiguous polynucleotide of equal length within the region of the SOD1 mRNA transcript having the nucleic acid sequence of any one of SEQ ID NOs: 33-48.

7. The inhibitory nucleic acid of claim 6, wherein the guide strand comprises from 15 to 21 contiguous nucleotides that are fully complementary to a contiguous polynucleotide of equal length within the region of the SOD1 mRNA transcript having the nucleic acid sequence of any one of SEQ ID NOs: 33-48.

8. The inhibitory nucleic acid of claim 7, wherein the guide strand comprises from 18 to 21 contiguous nucleotides that are fully complementary to a contiguous polynucleotide of equal length within the region of the SOD1 mRNA transcript having the nucleic acid sequence of any one of SEQ ID NOs: 33-48.

9. The inhibitory nucleic acid of claim 8, wherein the guide strand comprises 19, 20, or 21 contiguous nucleotides that are fully complementary to a contiguous polynucleotide of equal length within the region of the SOD1 mRNA transcript having the nucleic acid sequence of any one of SEQ ID NOs: 33-48.

10. The inhibitory nucleic acid of any one of claims 1-9, wherein the guide strand comprises 9 or fewer nucleotide mismatches relative to a segment of 15, 16, 17, 18, 19, 20, or 21 contiguous nucleotides within the region of the SOD1 mRNA transcript having the nucleic acid sequence of any one of SEQ ID NOs: 33-48, optionally wherein the guide strand comprises 8 or fewer, 7 or fewer, 6 or fewer, 5 or fewer, 4 or fewer, 3 or fewer, 2 or fewer, or only 1 mismatch relative to the region of the SOD1 mRNA transcript having the nucleic acid sequence of any one of SEQ ID NOs: 33-48.

11. The inhibitory nucleic acid of any one of claims 1-10, wherein the region of the SOD1 mRNA transcript has the nucleic acid sequence of any one of SEQ ID NOs: 33, 38, 40, 41, 42 and 45.

12. The inhibitory nucleic acid of any one of claims 1-11, wherein the guide strand has a nucleic acid sequence that is at least 85% identical to the nucleic acid sequence of any one of SEQ ID NOs: 1-16.

13. The inhibitory nucleic acid of claim 12, wherein the guide strand has a nucleic acid sequence that is at least 90% identical to the nucleic acid sequence of any one of SEQ ID NOs: 1-16.

14. The inhibitory nucleic acid of claim 13, wherein the guide strand has a nucleic acid sequence that is at least 95% identical to the nucleic acid sequence of SEQ ID NOs: 1-16, optionally wherein the guide strand has a nucleic acid sequence that is at least 96%, 97%, 98%, or 99% identical to the nucleic acid sequence of any one of SEQ ID NOs: 1-16.

15. The inhibitory nucleic acid molecule of claim 14, wherein the guide strand has the nucleic acid sequence of any one of SEQ ID NOs: 1-16.

16. The inhibitory nucleic acid of any one of claims 12-15 wherein the guide strand has the nucleic acid sequence of any one of SEQ ID NOs: 1, 6, 8, 9, 10 and 13.

17. The inhibitory nucleic acid of any one of claims 1-16, wherein the inhibitory nucleic acid comprises a hairpin having a nucleic acid sequence that is at least 85% identical to the nucleic acid sequence of any one of SEQ ID NOs: 17-32.

18. The inhibitory nucleic acid of claim 17, wherein the inhibitory nucleic acid comprises a hairpin having a nucleic acid sequence that is at least 90% identical to the nucleic acid sequence of any one of SEQ ID NOs: 17-32.

19. The inhibitory nucleic acid of claim 18, wherein the hairpin has a nucleic acid sequence that is at least 95% identical to the nucleic acid sequence of any one of SEQ ID NOs: 17-32, optionally wherein the hairpin has a nucleic acid sequence that is at least 96%, 97%, 98%, or 99% identical to the nucleic acid sequence of any one of SEQ ID NOs: 17-32.

20. The inhibitory nucleic acid of claim 19, wherein the hairpin has the nucleic acid sequence of any one of SEQ ID NOs: 17-32.

21. The inhibitory nucleic acid of any one of claims 17-20, wherein the hairpin has the nucleic acid sequence of any one of SEQ ID NOs: 17, 22, 24, 25, 26 and 29.

22. The inhibitory nucleic acid of any one of claims 1-21, wherein the inhibitory nucleic acid is an interfering RNA molecule, optionally wherein the interfering RNA molecule is a microRNA (miRNA), short hairpin RNA (shRNA), or short interfering RNA (siRNA).

23. The inhibitory nucleic acid of claim 22, wherein the inhibitory nucleic acid is a miRNA.

24. A viral vector comprising a transgene encoding the inhibitory nucleic acid of any one of claims 1-23, optionally wherein the viral vector comprises a plurality of the transgenes (e.g., 2, 3, 4, 5, or more of the transgenes).

25. The viral vector of claim 24, wherein the viral vector is selected from the group consisting of adeno-associated virus (AAV), adenovirus, lentivirus, retrovirus, poxvirus, baculovirus, herpes simplex virus, vaccinia virus, and a synthetic virus.

26. The viral vector of claim 25, wherein the viral vector is an AAV.

27. The viral vector of claim 26, wherein:
(i) the AAV is an AAV1, AAV2, AAV3, AAV4, AAV5, AAV6, AAV7, AAV8, AAV9, AAVrh10, or AAVrh74 serotype, or wherein the AAV comprises an AAV2-retro or an AAV9-retro capsid protein; and/or
(ii) the AAV comprises a capsid protein that is conjugated to a ligand or an aptamer.

28. The viral vector of claim 27, wherein the viral vector is a pseudotyped AAV.

29. The viral vector of claim 28, wherein the pseudotyped AAV is AAV2/9.

30. The viral vector of claim 28, wherein the pseudotyped AAV is AAV2/8.

31. The viral vector of claim 26, wherein the AAV comprises a recombinant capsid protein.

32. The viral vector of claim 25, wherein the synthetic virus is chimeric virus, mosaic virus, or pseudotyped virus, and/or comprises a foreign protein, synthetic polymer, nanoparticle, or small molecule.

33. A pharmaceutical composition comprising (i) the inhibitory nucleic acid of any one of claims 1-23 or the viral vector of any one of claims 24-32 and (ii) a pharmaceutically acceptable excipient, carrier, or diluent.

34. A method of treating a neurological disorder in a subject in need thereof, the method comprising administering to the subject a therapeutically effective amount of the inhibitory nucleic acid of any one of claims 1-23, the viral vector of any one of claims 24-32, or the pharmaceutical composition of claim 33.

35. The method of claim 34, wherein the neurological disorder is a neurodegenerative disorder.

36. The method of claim 34 or 35, wherein the neurological disorder is caused by, or associated with, expression of a wild-type or mutant form of SOD1.

37. The method of any one of claims 34-36, wherein the neurological disorder is amyotrophic lateral sclerosis (ALS), Huntington's disease, Parkinson's disease, frontotemporal lobar dementia, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy, dementia Parkinsonism ALS complex of guam, Pick's disease, Perry syndrome, cerebral age-related TDP-43 with sclerosis, hippocampal sclerosis, or Alzheimer's disease.

38. The method of claim 34, wherein the neurological disorder is ALS.

39. A method of treating a cell proliferation disorder in a subject in need thereof, the method comprising administering to the subject a therapeutically effective amount of the inhibitory nucleic acid of any one of claims 1-23, the viral vector of any one of claims 24-32, or the pharmaceutical composition of claim 33.

40. The method of claim 39, wherein the cell proliferation disorder is cancer.

41. The method of claim 40, wherein the cancer is leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, throat cancer, acute lymphoblastic leukemia, acute myeloid leukemia, chronic lymphocytic leukemia, chronic myelogenous leukemia, adrenocortical carcinoma, acquired immune deficiency syndrome-related lymphoma, primary central nervous system lymphoma, anal cancer, appendix cancer, astrocytoma, atypical teratoid rhabdoid tumor, basal cell carcinoma, bile duct cancer, extrahepatic cancer, Ewing's sarcoma, osteosarcoma, malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ, endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors, testicular germ cell tumor, gestational trophoblastic disease, glioma, childhood brain stem glioma, hairy cell leukemia, hepatocellular cancer, langerhans cell histiocytosis, Hodgkin's lymphoma, hypopharyngeal cancer, islet cell tumors, pancreatic neuroendocrine tumors, Wilms' tumor, childhood kidney tumors, nephroblastoma, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma, plasma cell neoplasms, plasmacytoma, myelodysplastic syndromes, nasopharyngeal cancer, neuroblastoma, non-Hodgkin's lymphoma, non-small cell lung cancer, epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, Kaposi sarcoma, Sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, or Waldenstrom's macroglobulinemia.

42. The method of any one of claims 34-41, wherein the inhibitory nucleic acid, viral vector, or pharmaceutical composition is administered to the subject by a route selected from intrathalamic, intrathecal, subpial, intraparenchymal, intrastriatal, intracranial, intracisternal, intracerebral, intracerebroventricular, intraocular, intravitreal, intraventricular, intralumbar, intravenous, intramuscular, subcutaneous, intraperitoneal, intradermal, transdermal, parenteral, intranasal, percutaneous, intratracheal, intraarterial, intravascular, and oral administration, inhalation, perfusion, lavage, or any combination thereof.

43. The method of any one of claims 34-42, wherein the subject is a mammal.

44. The method of claim 43, wherein the mammal is a human.

45. A kit comprising the inhibitory nucleic acid of any one of claims 1-23, the viral vector of any one of claims 24-32, or the pharmaceutical composition of claim 33, wherein the kit further comprises a package insert instructing the use of the kit to administer a therapeutically effective amount of the inhibitory nucleic acid, viral vector, or pharmaceutical composition to a subject, preferably wherein the subject is a human.
